(19) United States
(12) Patent Application Publication
(10) Pub. No.: US 2006/0282910 A1
(43) Pub. Date: Dec. 14, 2006

(54) Delphinium flower color crossing method

(76) Inventors: Fumio Hashimoto, Kagoshima (JP); Yusuke Sakata, Kagoshima (JP)

Correspondence Address: SQUIRE, SANDERS & DEMPSEY L.L.P., 14TH FLOOR, 8000 TOWERS CRESCENT, TYSONS CORNER, VA, 22182, US

(21) Appl. No.: 10/570,952

(22) PCT Filed: Aug. 20, 2004
(86) PCT No.: PCT/JP04/11979

§ 371 Date: Jun. 21, 2006

(30) Foreign Application Priority Data
Sep. 16, 2003 (JP) 2003-322518

(51) Int. Cl. A01H 1/02; C12N 5/02; G01N 37/00

(52) U.S. Cl. 800/266; 435/420; 702/81; 426/540

(57) ABSTRACT

It is intended to provide a flower color crossing method whereby a specific flower color is passed to the next generation of delphinium and a flower color crossing method whereby a dichromatic flower color is passed to the next generation of delphinium. It is also intended to provide a method whereby perpetual delphinium can be efficiently obtained in a warm place and a method of determining a delphinium flower color based on the ratio of main inherent colorants in the sepal. It is found out that a specific flower color can be passed to the next generation by using a full-color delphinium as a pollen parent or a seed parent and carrying out allogamous crossing and, at the same time, a dichromatic flower color can be passed to the next generation thereby. By growing a delphinium seedling under such conditions as allowing its germination in a petri dish at a temperature of about 15° C., a method of efficiently obtaining perpetual delphinium in a warm place is found out. By analyzing delphinium flower colors and inherent colorants, a numerical formula for determining a delphinium flower color is established. A crossing method for obtaining purple or pale purple delphinium flowers containing novel anthocyanin as the main component is found out. Also, a method of isolating and purifying the novel anthocyanin colorant is found out.

### TECHNICAL FIELD

The present invention relates to a method for effectively crossing delphinium in order to propagate seeds of delphinium. Also, the present invention relates to a method for effectively crossing delphinium in order to propagate seeds of bicolor type flower color delphinium. More specifically, the invention relates to novel plants and a treatment for obtaining the same comprising flowers of flowering plants, i.e., angiosperms and a crossing method which is a treatment for altering genotype. The present invention also includes a method for partially utilizing a breeding process containing sexual hybridization. The present invention is also directed to new plants, which are flowering plants such as (angiosperms and a method for obtaining the same.

### BACKGROUND ARTS

Delphinium is a herbaceous plant belonging to genus Delphinium, family Ranunculaceae. There are two hundreds or more kinds of delphinium in the world, and most of them are distributed in Europe, portions along the Mediterranean climate, Siberia, and California , which are the northern hemisphere (Non-Patent Document 1: Engei Shokubutsu Daijiten, Shogakukan, 348-350, 1989)

In Europe, delphinium wasd been dealt as a flower for a flower bed (perennial), but at the beginning of twenty century, selective breeding of delphinium was succeeded. At the present, in America, a group of variants having big, double flower such as Pacific Giant line and Dwarf Blue Fountain line delphiniums were provided, and in England and Holland, a group of variants having a single flower and having many branches such as Belladonna line and Pink Sensation line delphiniums were provided (Non-Patent Document 1, and Non-Patent Document 2: Asahi Engei Hakka 03, Asahi Newspaper, 204-206, 1984)

It is said that delphinium was introduced in Japan at early in Meiji, and recently, supplying of seeds and saplings of delphinium and selective breeding of delphinium fully grappled at last. Development of production methods has been forced including development of annually stable production (Non-Patent Document 3: Shigefumi Murakami, Research Center of Agriculture, Forestry, and Fishers, horticultural center at warm place, Horticultural Section, results of the year of Heisei 10, page 6, 1998), and forced production of delphinium at a warm place (Non-Patent Document 4: Kaoru Nakamura and 5 others, Miyazakiken, Sounou Shiken Gihou, 13-29, 1995). Amongst them, a group of varieties represented by “Sirius” can be assumed to be excellent F₁ variety suitable for forced cultivation at a warm place (Non-Patent 5: Hiroshi Nakamura, Miyazakiken, Sounou Shiken Gihou, 53-61, 2001)

In delphinium those reaching to height of approximately 1.5 m or more, and having 50 or more spikes (inflorescences) are assumed to be good. At a first sight, the spikes look like petals, but they are calyces, which are stained in blue, purple, pink yellow or white, creating various flower colors (Non-Patent Document 6: “Delphinium” Asahi Engei Hakka 06, Asahi Newspaper, 202-204, September, 1984).

As for anthocyanin pigments included in the calyces, it has been known that blue calyces include excessively acylated cyanodelphin (Non-Patent Document 7: Kondo, T, Tetrahedron Lett. 44:6375-6378, 1991). It has been clarified that purple calyces include violdelphin, which is acylated anthocyanin. It has been reported that these pigments are body for coloring calyces blue and purple (Non-Patent Document 8: Kondo, T., Chem. Lett., 137-138, 1990).

Flower color is sensitized by human's eye by exposing a light on the surface of petal and reflecting a light which has not been absorbed by pigments existing in epidermis of the petal. However, since there is a difference in sensitivity to light or to coloration among individuals, a method for clearly expressing flower color should be required (Non-Patent Document 9: Voss, D. H.: Hort Sci., 27:1256-1260, 1992).

As a method for measuring flower color, there is a measuring method wherein a calorimeter is used and the measured values are plotted on coordinates of CIELab calorimetric system. In this method, three color attributes, i.e., hue, brightness, and chroma, are considered as three dimensional global color chart, i.e., color i.e., as color cube, the hue difference in this space correctly reflects on the difference in color sensitized by naked eye (Non-Patent Document 10: Gonnet, J. F.: Food Chem.: 63:409-415, 1998).

In recent years, we reported a relation between flower color and endogenous pigment concentration (cyanodelphin and violdelphin) (Non-Patent Document 11: Hashimoto, F., J. Soc. Hort. Sci., 69:428-434, 2000). More specifically, it has reported that flower color of delphinium calyx is measured, and a relation with endogenous pigment obtained from the calyx is mentioned, whereby flower color can correctly measured.

Furthermore, we have reported that with regard to endogenous pigment concentration (cyanodelphin and violdelphin), the concentrations of them are changed with elapse of time after flowering, and have mentioned about biosynthesis thereof (Non-Patent Document 12: Hashimoto, F. Biosci. Biotechnol. Biochem., 66:1652-1659, 2002). More specifically, it has been mentioned that tulipanin obtained from the calyx is used as a starting pigment, which is changed into bisdeacylplatyconin, violdelphin, and cyanodelphin, with elapse of time. This biogenesis is not biosynthesis of antocyanidin as a core of anthocyanin, but concerns a biosynthesis which derives glycosidation and acylation of delphinidin which is an anthocyanidin.

As for coloration mechanism of calyx of blue type delphinium, it has been reported that existence of metal aluminum (Al³⁺) in vacuole of calyx endogenous cells is important, and copigmantation is brought about with cyanodelphin or such thereby bluing (Non-Patent Document 13: Hisami Yoshida, Summary of Nippon Nougei Kagakukai Nenkai, 262, 2002). It has been also reported that calyx endogenous cells is converted into protoplast to examine blue fibrous substance, as a result, any metal ion does not take part in bluing (Non-Patent Document 14: Hisami Yoshida, Summary of Nippon Nougei Kagakukai Nenkai, 263, 2003). Furthermore, in order to examine the coloration mechanism of blue type delphinium calyx. pH value of vacuole of calyx epithelial cell, it has been reported to be approximately 5.0 (Non-Patent Document 5, Hisami Yoshida et al., Nippon Shokubutsu Seirigakkai Nenkai Youshi. 277, 2001).

As for commercially available variations of delphinium, it has been said that they have ploidy of di-, tetra- and hexaploid. Pacific giant type varieties are tetraploid (Non Patent Document 16). Due to tetraploid, in the case of outbreeding crossking, progenies having various flower colors are obtained, leading to problem for having no process for inheriting a specific color to progeny. Amongst them, as a bicolor delphinium, ginever is selected, which is passed from mericlone seedling, and which is not propagated by propagation from seeds (Non-Patent Document 5). Consequently, seedlings propagated from seed cannot be freely obtained by crossing, various bicolor delphinium has not yet been provided to markets.

The delphinium is well growing at a cool or cold place or a high and cold place, and is not well growing at a warm place (Non-Patent Document 4). For this reason, the breeding of delphinium at seasons is avoided at a warm place. Consequently, there is a disadvantage that delphinium cannot be effectively cultivated unless it is forcedly cultivated.

Although the flower color of delphinium is correctly explained in numbers (Non-Patent Document 11), since relation between flower color of calyx and intrinsic pigment within the calyx is not clear, there is a disadvantage that flower color of delphinium cannot be decided from the deposition of intrinsic pigment.

In addition according to summary of lecture of the XXVIth International Horticultural Congress and Exhibition (Toronto Canada), three major anthocyanidins in Eustoma grandiflorum Cultivars are described (Non-Patent Document 17, Uddin, A. F. M. J.: the XXVIth International Horticultural Congress and Exhibition, 2002, August 11-17, P. 475-476).

We have applied the contents disclosed therein as Japanese Patent Application No. 2003-026598 (hereinafter referred to as Patent Document 4) entitled “Method for Crossing lisianthus based on genotype of its flower pigment” (paragraphs [0001] to [0019] of Patent Document 4). Patent Document 4 discloses that “considering heredity of three antocyanidins which are main flower pigments of lisianthus, pelargonidin (Pgn), cyaniding (Cyn), and delphinidin (Dpn), examinations have been made by performing self-pollination and reciprocal crossing, and as a result, new law of heredity has been found from separation of pigment phenotype of F₁ to F₃-progenies”, and that “four multiple allele, H^{T}, H^{F}, H^{D}, and H^{O}, exist in the enzymatic reaction systems of flavonoid 3′-hydroxylase (F3′H) and flavonoid 3′,5′-hydroxylase, (F3′,5′H) contributing hydroxylation of B ring of pigment precursor, and they control hydroxylation at 3′-position, 5′-position, 3′,5′-position, and 3′-and 5′-positions”.

Japanese Patent Laid-Open H11-103704 (hereinafter referred to as Patent Document 2) discloses a method for obtaining F₁ seeds utilizing mericlone petal obtained by propagating tissue cultured petal. Specifically, there is description that “a method is characterized in that at least one individuals selected from selected individuals of two self-propagation delphinium line is tissue-cultivated and propagated, the resulting one mericolone petal and the other self-propagated individual or both of mericlone petals are crossed to obtain F1 seed of delphinium (claim 1 of Patent Document 2).

Japanese Patent Laid-Open No. H11-032604 (hereinafter referred to as Patent Document 3) discloses a method for producing petals crossed between variants. Specifically, there is description that the gist of the present invention is a method for producing petals crossed between variants of delphinium characterized in the fact that embryo obtained by crossing between variants of plants belonging to delphinium is extracted or at least part of the resulting embryo is exposed to use embryo cultivation (0008 paragraph of Patent Document 3).

U.S. Pat. No. 13,010 (hereinafter referred to as Patent Document 4) discloses a novel horticultural variety of delphinium, “Dolce Vita”, which is described to have double flower and blue bicolor flower (0010 paragraph of Patent Document 4).

U.S. Pat. No. 14,152 (hereinafter referred to as Patent Document 5) discloses a novel horticultural variety of delphinium, “Delga Stam”. According to Patent Document 5, paragraph 0011, the present invention relates to a novel horticultural variety belonging to delphinium, which is botanically crossed variant whose flower color is blue purple/pale green.
Patent Document 1: Japanese Patent Application No. 2003-026598 [symbol] (0015 paragraph)
Patent Document 2: Japanese Patent Laid-Open H 11-103704 (claim 1)
Patent Document 3: Japanese Patent Laid-Open No. H11-032604 (0008 paragraph)
Patent Document 4: U.S. Pat. No. 13,010 (0010 paragraph)
Patent Document 5: U.S. Pat. No. 14,152 (0004 and 0011 paragraphs)
Non-Patent Document 1: “Genus Delphinium” Engei Shokubutsu Daijiten, Shogakukan, 348-350, 1989
Non-Patent Document 2: “Delphinium” Asahi Engei Hakka 03, Asahi Newspaper, 204-206, June, 1984)
Non-Patent Document 3: “Development of Annually Stable Production of Delphinium”, Shigefumi Murakami, Research Center of Agriculture, Forestry, and Fishers, horticultural center at warm place, Horticultural Section, results of the year of Heisei 10, page 6, 1998)
Non-Patent Document 4: “Establishment Technique for Forced Production of Delphinium at Warm Place”, Kaoru Nakamura and 5 others, Miyazakiken, Sounou Shiken Gihou, 13-29, July, 1995)
Non-Patent Document 5: Hiroshi Nakamura, and 6 others, “Raising and Characteristics of Delphinium F₁ variety Sirius suitable for Forced Breeding at Warm Place”, Miyazakiken, Sounou Shiken Gihou, 53-61, March, 2001)
Non-Patent Document 6: “Delphinium” Asahi Engei Hakka 06, Asahi Newspaper, 202-204, September, 1984)
Non-Patent Document 7: Kondo, and 6 Others, “Structure of Cyanodelphin, a Tetra-p-hydroxybenzoated Anthocyanin from Blue Flower of Delphinium hybridum”, Tetrahedron Lett, 1991, Vol. 44, P. 6375-6378
Non-Patent Document 8: Kondo, and 3 Others, “Structure of Violdelphin, an Anthocyanin from Violet Flower of Delphinium hybridum”, Chem. Lett, 1990, P. 137-138
Non-Patent Document 9: Voss, D. H., “Colorimeter Measurement of Plant Color to the Royal Horticultural Society Colour Chart, Hort Sci., 1992 Vol. 27, P. 1256-1260
Non-Patent Document 10: Gonnet, J. F. “Color Effects on Co-pigmentation of Anthocyanins revisited. 1. A Colorimetric Definition Using the CIELAB Scale, Food Chem., 1998, Vol. 63, P. 409-415
Non-Patent Document 11: Hashimoto, F. and 4 Others, “Characterization of Cyanic Flower Color of Delphinium Cultivars”, J. Soc. Hort. Sci., 2000, Vol. 69, P. 428-434
Non-Patent Document 12: Hashimoto, F. and 5 others, “Changes of Flower Coloration and Sepal Anthocyanins of Cyanic Delphinium Cultivars during Flowering”, Biosci. Biotechnol. Biochem., 2002, Vol. 66, P. 1652-1659
Non-Patent Document 13: Hisami Yoshida, “Mechanism of Coloration of Blue Delphnium Patal”, Summary of Nippon Nougei Kagakukai Nenkai, 262, 2002
Non-Patent Document 14: Hisami Yoshida, “Mechanism of Coloration of Blue Delphinium Patal 2—Analysis of Blue Color Substances in Vacuole”, Summary of Nippon Nougei Kagakukai Nenkai, P 263, 2003
Non-Patent Document 15: Hisami Yoshida et al., Nippon Shokubutsu Seirigakkai Nenkai Youshi. 277, 2001)
Non-Patent Document 16: Legro, R. A. H., Euphytica, [Species Hybrids in Delphinium], Euphytica, 1961, Vol 10, p. 1-23.
Non-Patent Document 17: Uddin, A. F. M. J., and 2 others [Inheritance Model of Three Major Anthocyanidins in Eustoma grandiflorum Cultivars], On-Site Program, the XXVIth International Horticultural Congress and Exhibition, Toronto, Canada, 2002, August 11-17, P. 475-476(S19-P-19).

### SUMMARY OF THE INVENTION

However, delphinium is an allogamous plant and thus, causes self-propagation weakness when repeating self-propagation. Consequently, since it is difficult to propagate seeds having a specific flower color, seeds having specific flower color cannot be maintained. Furthermore, there is a disadvantage that when allogamous crossing is conduced with delphinium progenies having various flower colors are obtained, making it impossible to inherit a specific flower color to a progeny.

Bicolor delphinium can be passed and propagated from mericlone seedling, but since no seed propagation can be done, various bicolor delphinium has not yet been provided to markets.

The delphinium is well growing at a cool or cold place or a high and cold place, and is not well growing at a warm place (Non-Patent Document 4). For this reason, the breeding of delphinium at seasons is avoided at a warm place. Consequently, there is a disadvantage that delphinium cannot be effectively cultivated unless it is forcedly cultivated.

Although the flower color of delphinium is correctly explained in numbers, since relation between flower color of calyx and internal intrinsic pigment within the calyx is not clear, there is a disadvantage that flower color of delphinium cannot be decided from the deposition of intrinsic pigment.

Calyx of delphinium contains anthocyanin pigments having unknown chemical structure, delphinium having purple flower possessed by these pigment cannot be put into market.

An object of the present invention is to provide a method for obtaining seeds having a specific flower color from all color type delphinium allogamous crossing, as well as to provide a crossing method for obtaining seeds of delphinium having bicolor type flower color from allogamous crossing. Another object of the present invention is to provide a cultivation method for cultivating delphinium at season at a warm place.

An object of the present invention is also to provide a method for freely obtaining seeds having a specific flower color from allogamous crossing. Also, an object of the present invention is to provide various bicolor delphiniums from seed propagation.

Moreover, an object of the present invention is to provide delphinium having purple or pale purple flower containing a novel anthocyanin as a main component and to provide an isolation method and a purification method of the novel anthocyanin.

In order to attain these object, we have found that delphinium having whole color type flower color can be used as a pollen parent or a seed parent to allogamous crossing to thereby allow a specific flower color to be inherited to a progeny.

Furthermore, it has been found that delphinium as a pollen parent or a seed parent to allogamous crossing to thereby allow a bicolor flower color to be inherited to a progeny.

Moreover, we have found that delphinium having whole color type flower color can be used as a pollen parent or a seed parent to allogamous crossing to thereby allow a specific flower color to be inherited to a progeny.

In addition, when raising seedling under the conditions that delphinium is germinated within a laboratory dish at a temperature of 15° C.±1° C. (14-16° C., hereinafter referred to as approximately 15° C.), delphinium can be effectively growing (at a season), and particularly at a warm place.

A method for determining flower color of delphinium has been found which comprising applying an equation 1:

$$H = {H}_{max} \frac{[ CD / VD ]}{[ CD / VD ] + {K}_{H}} = {tang}^{- 1} ( {b}^{*} / {a}^{*} ) \qquad ( 1 )$$

where ratio of content of intrinsic pigment within the calyx [CD/VD] is taken as a horizontal axis and hue angle exhibiting flower color wherein CD/VD exhibits ratio of major intrinsic pigment within the calyx of delphinium ; CD is Cyanodelphin, VD is of Violdelphin, Hmax is the maximum hue angle showing flower color, and K_{H} is a constant of ratio of intrinsic pigment in the case of half of the maximum, hue angle.

A method for crossing delphinium according to the present invention I to determine the combination of flower color crossing for creating flower color of delphinium, which assumes flower color utilizing a simplified chart taking gamete of pollen parent or seed parent as a line or column.

In the method for crossing delphinium according to the present invention, the predetermined anthocyanin pigment is a novel anthocyanin pigment (I) represented by the following formula (I) or an anthocyanin pigment (I) represented by the following formula (II):

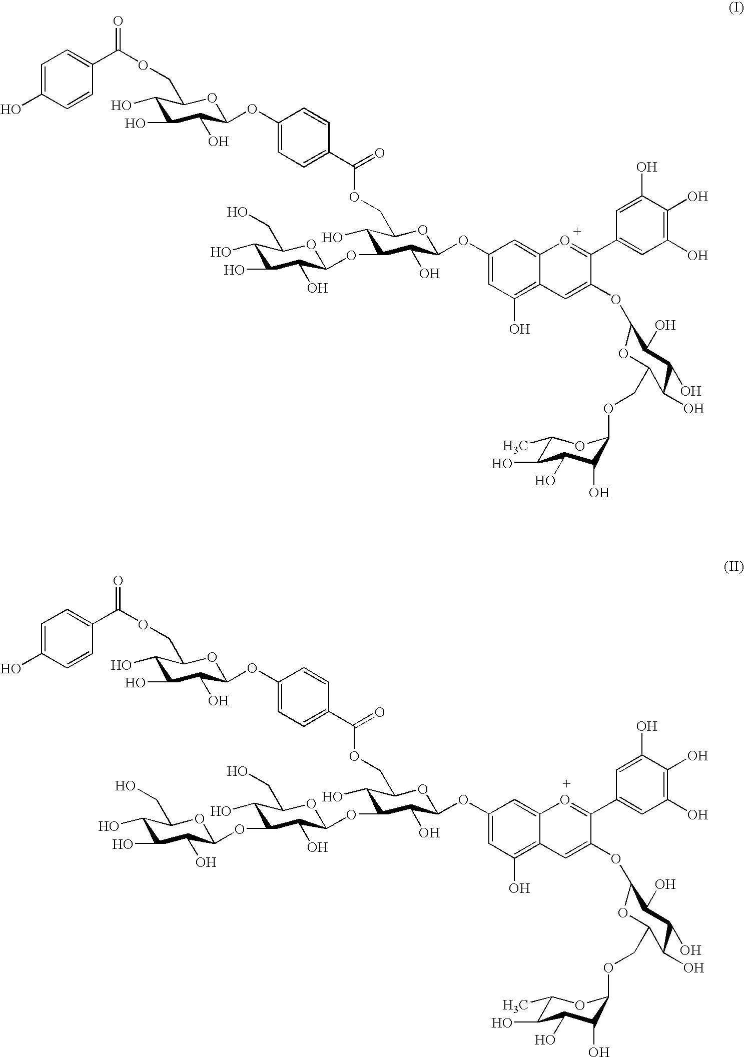

These pigments are a novel or not isolated from delphinium. Consequently, the present invention includes a method for extracting an anthocyanin pigment which comprises isolating anthocyanin pigment (I), anthocyanin pigment (II), or both anthocyanin pigments them from the calyx of delphinium obtained by the method. The method for crossing delphinium according to claim 8 or 9, followed by purifying. Also, included herein is a novel compound,
3-O-(6-O-(α-L-rhamnosyl)-β-D-glucopyranosyl)-7-O-(3-O-β-D-glucopyranosyl-6-O-(4-O-(6-O-p-hydroxybenzoyl-β-D-glucopyranosyl)-p-hydroxybenzoyl)-β-D-glucopyranosyl)-delphinidin, represented by the following formula (I).

### BRIEF DESCRIPTION OF THE DRAWINGS

FIG. 1 is a drawing which plots bicolor flower color on the coordinates of CIELab standard calorimetric system (Example 5)

FIG. 2 is a photo showing the shape of organism of bicolor blue: B blue (Example 5).

FIG. 3 is a photo showing the shape of organism of bicolor light blue, B light blue (Example 5).

FIG. 4 is a drawing showing change in hue angles of Pacific Giant (purple) and Blue Mirror (Blue) with time elapse (Example 7).

FIG. 5 is a drawing showing the relation between major intrinsic pigment ratio of the calyx of Pacific Giant (CD/TP) and major intrinsic pigment ratio of the calyx of Blue Mirror (CD/VD) relative to hue angle (Example 7).

FIG. 6 is a drawing showing relation between a reciprocal number of major intrinsic pigment ratio of the calyx of Pacific Giant (CD/TP) and a reciprocal number of major intrinsic pigment ratio of the calyx of Blue Mirror (CD/VD) relative to a reciprocal number of hue angle (Example 7).

FIG. 7 is a drawing which compares revival ratio of hybrid seed obtained by to allogamous crossing of Pacific Giant with that of purchased seed (shop variety) (Example 10).

FIG. 8 is a drawing of nuclear magnetic resonance] spectrum of novel anthocyanin pigment represented by formula (I) showing hetronuclear chemical shift correlation (FG-HMQC) and homonuclear chemical shift correlation (curve shown by arrow ¹H-¹H TOCOSY) of novel anthocyanin pigment (Example 12).

FIG. 9 is a drawing of nuclear magnetic resonance] spectrum of novel anthocyanin pigment represented by formula (I) showing (FG-HMBC) of 13 Carbon (¹³C) correlating with proton (¹H) (Example 12).

### BEST MODE FOR CARRYING OUT THE INVENTION

Embodiments of the present invention will now be described.

The present invention relates to a method for crossing delphinium based on flower color comprising

utilizing delphinium having whole color type flower color as a pollen parent or a seed parent to allogamous crossing to thereby allow a specific flower color to be inherited to a progeny.

More specifically, as for Pacific Giant, a blue color flower can be obtained by allogamous crossing of blue color flower with light blue color flower, a white color flower can be obtained by allogamous crossing of blue color flower with white color flower, a purple flower can be obtained by allogamous crossing of blue color flower with purple color flower or purple color flower with pale purple flower, and a pale purple flower can be obtained by allogamous crossing of pale purple color flower with white color flower.

Also, as for Blue Springs, a blue color flower and a light blue color flower can be obtained by allogamous crossing of blue color flower with light pale purple color flower, a white color flower can be obtained by allogamous crossing of light blue color flower with light white color flower, a purple color flower can be obtained by allogamous crossing of purple color flower with pale purple color flower or pale purple flower with pink color flower, and a red pink flower can be obtained by allogamous crossing of pink color flower with red pink color flower.

Moreover, it has been found that delphinium as a pollen parent or a seed parent can be used for allogamous crossing to thereby allow a bicolor flower color to be inherited to a progeny. More specifically, Pacific giant, which has light blue flower, and Blue Springs, which has red pink flower can be used as pollen parent or seed parent, and crossed to obtain a bicolor blue (B, Blue) in which the outside of calyx is blue and inside of calyx is purple can be obtained.

As for Pacific Giant, light blue color flower and pale purple color flower are used as pollen parent or seed parent to undergo allogamous crossing to thereby obtain bicolor flower (bicolor blue; B Blue) and bicolor light blue (B light Blue) in which outside of calyx is light blue and inside of calyx is light purple, and light blue color flower and pale purple color flower are used as pollen parent or seed parent to undergo allogamous crossing to thereby obtain bicolor, and a light blue color flower and a white color flower are used as pollen parent or seed parent to undergo allogamous crossing to thereby obtain bicolor flower (bicolor blue, B light blue).

As for Blue Springs, blue color flower and red pink color flower are used as pollen parent or seed parent to undergo allogamous crossing to thereby obtain bicolor flower (bicolor blue, B blue), light blue color flower and pale purple color flower are used as pollen parent or seed parent to undergo allogamous crossing to thereby obtain bicolor flower (bicolor light blue, B light blue), light blue color flower and red pink color flower are used as pollen parent or seed parent to undergo allogamous crossing to thereby obtain bicolor flower (bicolor blue, B blue),

blue color flower and purple color flower are used as pollen parent or seed parent to undergo allogamous crossing to thereby obtain bicolor blue and bicolor light blue.

As described above, various delphiniums having whole color type undergo allogamous crossing to thereby allow a specific flower color, especially bicolor flower color, to be inherited to a progeny.

Delphiniums which can be used in the method for crossing delphinium based on flower color, are not specifically restricted, but include, D. cheilantum, D. cardinale, D. consolida, D. elatum, D. grandiflorum, D. nudicaule, D. zalil, D. tatsienense, D. parryi, D. trolliifolium, D. nuttallianum, D. virescens, D. tricorne, D. bicolor, D. barbeyi, D. dubium, D. anthriscifolium, D. lacostei, D. macrocentron, and D. caeruleum.

Also, delphinium hybrid, which can be used in the method for crossing delphinium based on flower color, are not specifically restricted, but include, Black Night, Blue Bird, Gelahad, Guenevere, King Arthur, Percival, Summer Skies, Sir Lancelot, Baby Doll, Blue Nile, Butterball, Purple Shade, Ronald Watts, Dwarf Pacific, Dwarf Delphinium, Little Delphinium, Magic Fountain, Snow White, Blue Springs, Blladonna, Blue Bee, Moerheimii, Pink Sensation, Wendy, University Hybrid, Princess Caroline, Royal Red, Royal Yellow, Summer Dream, Sunkist, Sky Rocket, Beavery Hills Scarlet, Beavery Hills Yellow Shade, Beavery Hills Salmon Shade, Swan Lake, and Blue Pearl.

We have found that thus crossed delphinium can be effectively growing (at season) by raising seedling under the conditions that the delphinium is germinated within a laboratory dish at a temperature of 15° C.±1° C. Especially, in conventional it is difficult to allow delphinium for effectively growing at a warm place, but the present invention makes it possible to effectively growing at a warm place by germinating such conditions.

More specifically, seeds of delphinium (including bicolor flower color) which have undergone allogamous crossing are germinated within a laboratory dish at a temperature of approximately 15° C. In comparison with those in which commercially available seeds are germinated under the same conditions, examining final revival ratio (flowering ratio), approximately 0 to 15% of revival ratio (flowering ratio) was obtained for commercially available seeds, while approximately 15 to 28% of revival ratio (flowering ratio) was obtained for allogamous crossed seed, which is of significance. As a result, when the allogamous crossed seeds are raising under the conditions they are germinated within a laboratory dish at a temperature of approximately 15° C., the revival ratio at a warm place can be increased.

It has been found that when the flower color is determined in the method for crossing delphinium based on flower color according to the present invention, when an equation 1:

$$H = {H}_{max} \frac{[ CD / VD ]}{[ CD / VD ] + {K}_{H}} = {tang}^{- 1} ( {b}^{*} / {a}^{*} ) \qquad ( 1 )$$

where ratio of content of intrinsic pigment within the calyx [CD/VD] is taken as a horizontal axis and hue angle exhibiting flower color wherein CD/VD exhibits ratio of major intrinsic pigment within the calyx of delphinium; CD is Cyanodelphin, VD is of Violdelphin, Hmax is the maximum hue angle showing flower color, and K_{H} is a constant of ratio of intrinsic pigment in the case of half of the maximum, hue angle,
is used, flower color can be effectively determined.

Flower color of delphinium is change with time elapse after flowering. The reciprocal number of hue angle (h) is taken on the vertical axis. If the major pigment relating to flower color is blue color flower and light blue color, the pigment is changed from violdelphin (VD) to cyanodelphin (CD) with the elapse of time, and if it is purple color flower and pale purple flower, the pigment is changed from tulipanin (TP) to violdelphin (VD). The concentration of major pigment of the major intrinsic pigment before change is taken as denominator and that after the change is taken as numerator, and the reciprocal number of the concentration ratio is taken as the horizontal axis. Specifically, the concentration ratio of major intrinsic pigment of blue color flower and light blue color flower is calculated as cyanodelphin concentration/violdelphin concentration. This is indicated as [CD/VD]. On the other hand, the concentration ratio of major intrinsic pigment of purple color flower and pale purple flower is indicated as violdelphin concentration/tulipanin concentration. This is indicated as [VD/TP].

Taking a reciprocal number of hue angle (h) as the vertical axis, and a reciprocal number of concentration ratio of major intrinsic pigment, relation between them cam be indicated as a tropics equation, i.e., represented by formula 1 or formula 2.

$$H = {H}_{max} \frac{[ CD / VD ]}{[ CD / VD ] + {K}_{H}} = {tang}^{- 1} ( {b}^{*} / {a}^{*} ) \qquad ( 1 )$$

wherein CD/VD exhibits ratio of major intrinsic pigment within the calyx of delphinium; CD is Cyanodelphin, VD is of Violdelphin, Hmax is the maximum hue angle showing flower color, and K_{H} is a constant of ratio of intrinsic pigment in the case of half of the maximum, hue angle.

$$\frac{1}{H} = \frac{1}{{H}_{max}} + \frac{{K}_{H}}{{H}_{max}} \cdot \frac{1}{[ CD / VD ]} \qquad ( 2 )$$

From the formula, the maximum hue angle (Hmax) can be obtained. The maximum hue angle (Hmax) intended herein is a hue angle on CIELab calorimetric system of flower color when flower color of delphinium which will changes accompanying with flowering, is matured after flowering and is stabilized. In formulae 1 and 2, K_{H} means a constant of concentration ratio, and is a concentration ratio of major intrinsic pigment in the case of half of the maximum, hue angle. A method for determining flower color of delphinium has been found from formulae 1 and 2.

Calyx (flower) of blue type delphinium (blue, light blue, purple, pale purple color delphinium) is collected, and anthocyanin pigment is extracted with a solution (50% acetic acid/methanol) composed of 1:1 mixture of acetic acid with ethanol. The extracted solution is filtrated through a cotton plug, and then the solvent is distilled out under a reduced pressure by a rotary evaporator. The extracted residue is dissolved in an aqueous 5% acetic acid solution, and is subjected to an open column chromatography. The conditions of the open column chromatography are as follows: As stationary layer, MCI gel CHP-20P (CHP-20P, Mitsubishi Chemical Corporation), Sephadex LH-20 (Pharmacia Biotech), (Chromatorex ODS, Fuji Silysia Chemical LTD.) are used. As mobile layer, an aqueous 5% acetic acid solution as A liquid and an aqueous 5% acetic acid-methanol solution as B liquid are used. By increasing the contents from A liquid to B liquid, various chromatographic tests are conducted. As a stationary phase, Sephadex LH-20 (Pharmacia Fine Chemical) is used, as for mobile phase, an aqueous 5% acetic acid solution is used as A liquid, and an aqueous 5% acetic acid-acetone solution is used as C liquid. By increasing the contents from C liquid to A liquid, various chromatographic tests are conducted. By repeating these open column chromatographic tests, it has been found that novel anthocyanin pigment represented by formula (I) and anthocyanin pigment represented by formula (II) can be isolated.

When raising seedling under the conditions that the delphinium described above is germinated within a laboratory dish at a temperature of 15° C.±1° C. delphinium can be effectively growing (at a season), and particularly at a warm place.

When anthocyanin of calyx of self-pollination Pacific Giant is examined, it has been found that it contains novel anthocyanin pigment (I), and has anthocyanin pigment (II) as main pigment. By allogamous crossing of Pacific Giant having blue color flower with Pacific Giant having pale purple color flower, Pacific Giant having purple color flower and Pacific Giant having pale purple color flower containing novel anthocyanin pigment (I) and containing anthocyanin pigment (II) as main pigment can be obtained. By allogamous crossing of Pacific Giant having blue color flower with Pacific Giant having white color flower, Pacific Giant having purple color flower containing anthocyanin pigment (II) as main pigment can be obtained. By allogamous crossing of Pacific Giant having blue color flower with Pacific Giant having white color flower, Pacific Giant having pale purple color flower containing novel anthocyanin pigment (I) and containing anthocyanin pigment (II) as main pigment can be obtained.

By allogamous crossing of Blue Springs having light blue color flower with Blue Springs having pale purple color flower, Blue Springs having bicolor flower (light blue, B light blue) containing novel anthocyanin pigment (I) in the outside calyx and containing anthocyanin pigment (II) as main pigment can be obtained. By allogamous crossing of Blue Springs having light blue color flower with Blue Springs having white color flower, Blue Springs having bicolor flower (blue, B blue) containing novel anthocyanin pigment (I) in the inside calyx and containing anthocyanin pigment (II) as main pigment can be obtained. By allogamous crossing of Blue Springs having pale purple color flower with Blue Springs having red pink color flower, Blue Springs having purple color flower containing novel anthocyanin pigment (I) and containing anthocyanin pigment (II) as main pigment can be obtained. By allogamous crossing of Blue Springs having white color flower with Blue Springs having blue color flower, Blue Springs having pale purple containing novel anthocyanin pigment (I) and containing anthocyanin pigment (II) as main pigment can be obtained. Based on these findings of obtaining hybrid variety of delphinium, the present invention has been accomplished.

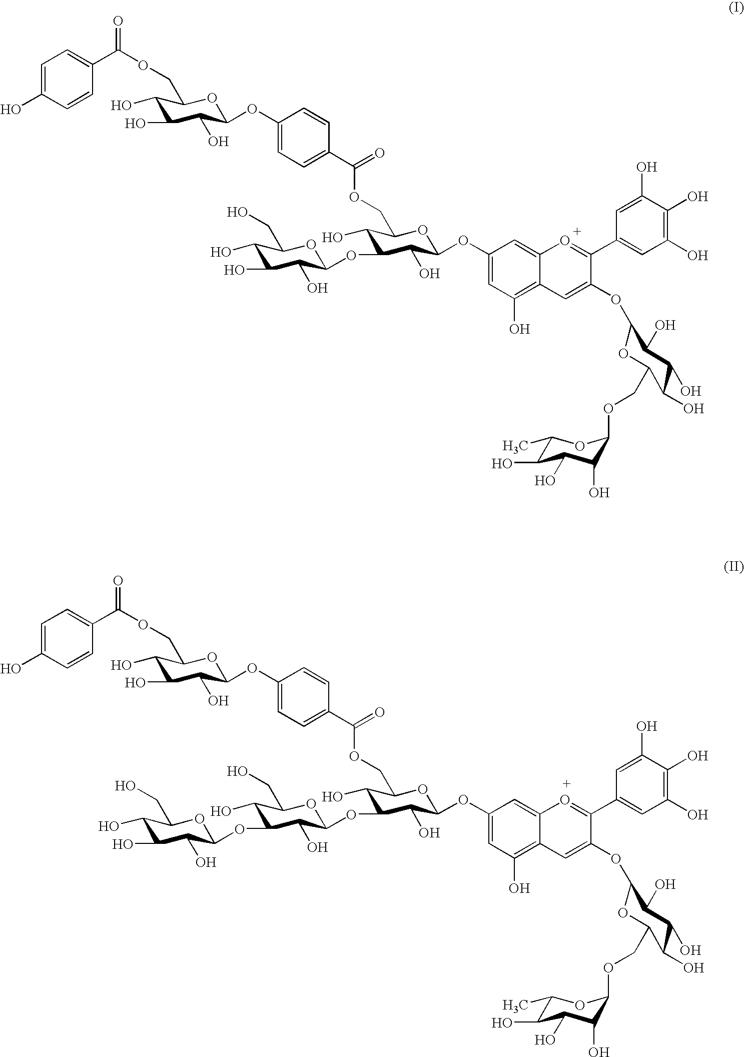

The method for crossing delphinium based on flower color according to the present invention comprises: utilizing delphinium having whole color type flower color as a pollen parent or a seed parent to allogamous crossing to thereby allow a specific flower color to be inherited to a progeny, the method including utilizing delphinium as a pollen parent or a seed parent to allogamous crossing to thereby allow a bicolor flower color to be inherited to a progeny

The present invention includes a method for cultivating delphinium comprising raising seedling under the conditions that delphinium is germinated within a laboratory dish at a temperature of 15° C.±1° C. (14-16° C.).

The present invention includes a method for cultivating delphinium comprising raising seedling under the conditions that delphinium is germinated within a laboratory dish at a temperature of 15° C.±1° C.

4. A method for determining flower color of delphinium which comprising applying an equation 1:

$$H = {H}_{max} \frac{[ CD / VD ]}{[ CD / VD ] + {K}_{H}} = {tang}^{- 1} ( {b}^{*} / {a}^{*} ) \qquad ( 1 )$$

where ratio of content of intrinsic pigment within the calyx [CD/VD] is taken as a horizontal axis and hue angle exhibiting flower color wherein CD/VD exhibits ratio of major intrinsic pigment within the calyx of delphinium; CD is Cyanodelphin, VD is of Violdelphin, Hmax is the maximum hue angle showing flower color, and K_{H} is a constant of ratio of intrinsic pigment in the case of half of the maximum, hue angle.

The method for crossing delphinium based on flower color according to the present invention include the method for determining flower color of delphinium, which applies an equation [VD/TP] to [CD/VD]

wherein [VD/TP] is a concentration ratio of major intrinsic pigment, and calculated by dividing Violdelphin concentration VD by tulipanin concentration TP.

The method for crossing delphinium based on flower color according to the present invention determines the combination of flower color crossing for creating flower color of delphinium, which assumes flower color utilizing a simplified chart taking gamete of pollen parent or seed parent as a line or column.

In the method for crossing delphinium according to the present invention, the predetermined anthocyanin pigment is a novel anthocyanin pigment, 3-O-(6-O-(α-L-rhamnosyl)-β-D-glucopyranosyl)-7-O-(3-O-β-D-glucopyranosyl-6-O-(4-O-(6-O-p-hydroxybenzoyl-β-D-glucopyranosyl)-p-hydroxybenzoyl)-β-D-glucopyranosyl)-delphinidin represented by the following formula (I); 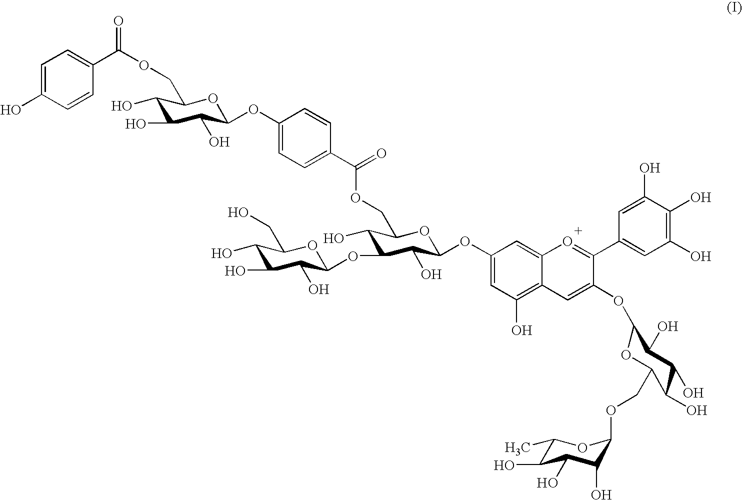
A novel anthocyanin pigment comprises 3-O-(6-O-(α-L-rhamnosyl)-β-D-glucopyranosyl)-7-O-(3-O-β-D-glucopyranosyl-6-O-(4-O-(6-O-p-hydroxybenzoyl-β-D-glucopyranosyl)-p-hydroxybenzoyl)-β-D-glucopyranosyl)-delphinidin, represented by the following formula (I):

The present invention includes the method for crossing delphinium, wherein the predetermined anthocyanin pigment is an anthocyanin pigment, 3-O-(6-O-(α-L-rhamnosyl)-β-D-glucopyranosyl)-7-O-(3-O-(3-O-(β-D-glucopyranosyl)-β-D-glucopyranosyl)-6-O-(4-O-(6-O-p-hydroxybenzoyl-β-D-glucopyranosyl)-p-hydroxybenzoyl)-β-D-glucopyranosyl)-delphinidin(II) represented by the following formula (II):

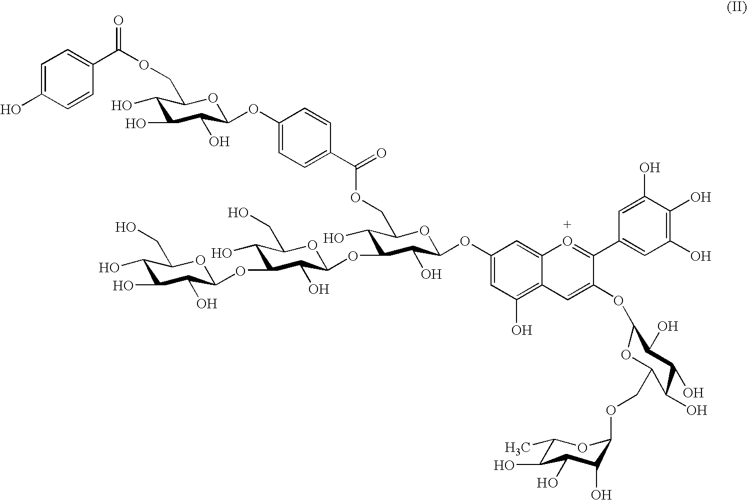

An anthocyanin pigment is represented by 3-O-(6-O-(α-L-rhamnosyl)-β-D-glucopyranosyl)-7-O-(3-O-(3-O-(β-D-glucopyranosyl)-β-D-glucopyranosyl)-6-O-(4-O-(6-O-p-hydroxybenzoyl-β-D-glucopyranosyl)-p-hydroxybenzoyl)-β-D-glucopyranosyl)-delphinidin(II).

According to the present invention, the above-mentioned novel anthocyanin pigment (I) and/or anthocyanin pigment (II) can be isolated and purified from calyx of delphinium.

### EXAMPLES

The present invention will now be described in more detail by referring to the working example, but the present invention is not restricted to these examples.

### Example 1

### Cultivation Method Raising Seedling Under the Conditions that Delphinium is Germinated Within a Laboratory Dish at a Temperature of Approximately 15° C.

Pacific Giant underwent allogamous crossing at spring of 1999 to obtain F₁ seeds. At the middle of August 2000, F₁ seeds were seeded within a laboratory dish. Defatted cotton were previously placed within the laboratory dish, and water was absorbed thereon in such an seeds were half sunk within the defatted cotton. The laboratory dish within which seeds were seeded was placed within a refrigerator at 15° C. for about 7 days to 10 days under dark conditions to germinate seeds. The germinated seeds were planted on a cell tray one after another. The planted cell tray was cultivated within a greenhouse at a high temperature of from 25 to 32° C., and seedlings were transferred to vinyl house. For promoting growth, heating through a heater was started from the last ten days of December to keep the temperature of the vinyl house at approximately 15° C. In order to accelerate differentiation of flower bud, long day treatment with lighting was conducted from the middle ten day of January 2001 to the last ten days of April 2001. Lighting conditions were as follows: Height from ridge was 1.1 m, one 100 W incandescent lamp per an area of 9 m², lighting over a period of 6 hours from 9:00 p.m. to 3:00 a.m. Number of seeds seeded within the laboratory dish was counted, and number of delphinium which was flowering from April to May, 2001 was counted to calculate revival (flowering) ratio.

In comparison, self-propagation seeds were similarly seeded within the laboratory dish to calculate revival (flowering) ratio.

The cultivation method was repeated three times, at August 2001 and August 2002, and the number of delphinium flowering from 2001 to 2003 was examined.

On the other hand, for comparison, purchased seeds were seeded within the laboratory dish at total twice of August 2001 and August 2001, and the commercially available seeds were cultivated to calculate revival (flowering) ratio.

Moreover, similar test were conducted for Blue Springs.

Furthermore, as for F₁ seeds obtained by allogamous crossing of Pacific Giant with Blue Springs, similar test examinations were conducted. The results are shown in Table 1.

TABLE 1

| | Flowering ratio | | |
|---|---|---|---|
| | (%) at each year | | |
| Kind of Delphinium Seed | 2000 | 2001 | 2002 |
| Pacific Giant (Commercial) | — | 0 | 14.9 |
| Pacific Giant (self-propagation) | 14.3 | 9.1 | 7.5 |
| Pacific Giant (allogamous crossing) | 22.3 | 28.4 | 16.0 |
| Blue Springs (Commercial) | — | 0 | 0 |
| Blue Springs (self-propagation) | 3.6 | 11.4 | 1.1 |
| Blue Springs (allogamous crossing) | 17.9 | 11.4 | 30.6 |
| Pacific Giant × Blue Springs | 23.0 | 15.6 | 30.0 |
| (allogamous crossing) | | | |

As is clear from Table 1, seedlings from F₁ seeds obtained by allogamous crossing were of highest revival ratio.

### Example 2

Pacific Giant was used as pollen parent or seed parent to conduct allogamous crossing to obtain F₁ seeds. The F₁ seeds were cultivated on the laboratory dish under the condition they were germinated at approximately 15° C., the seedlings were cultivated, were flowering, and their progenies were examined. The results are shown in Table 2.

TABLE 2

| | | Pacific Giant | | | | |
|---|---|---|---|---|---|---|
| | | Blue | L. Blue | Purple | P. Purple | White |
| Pacif- | Blue | B 7(78) | | | | |
| ic | | We 2(22) | | | | |
| Giant | L. Blue | B 8(89) | | | | |
| | | P 1(11) | | | | |
| | Purple | P 12(86) | P. 7(39) | P 5(83) | | |
| | | B 1(7) | B. 2(11) | P.P 1(16) | | |
| | | B b 1(7) | B.b 8(44) | | | |
| | | | B.lb 1(5) | | | |
| | Pale | P.P8(67) | W. 1(11) | P 9(100) | P.P 8(100) | |
| | Purple | P. 3(25) | B.lb 7(78) | | | |
| | | B. b 1(8) | B.l 1(11) | | | |
| | White | W. 15(56) | L.B 4(50) | P 4(36) | P.P | White |
| | | B. 4(15) | Blb 4(50) | PP 4(36) | 10(100) | 1(100) |
| | | P.P 3(11) | | Blb 2(18) | | |
| | | P. 1(9) | | Bb 1(9) | | |
| | | B.b 2(7) | | | | |
| | | B.lb 2(7) | | | | |

B: Blue,
LB: Light Blue
P: Purple
PP: Pale Purple
W: White
Bb: B Blue
Blb: B Light Blue

As shown in Table 2, blue color flower and light blue color flower were used as pollen parent or seed parent to conduct allogamous crossing, whereby 8 individuals of blue color flower and one individual of purple color flower were obtained. Blue color flower and white color flower were used as pollen parent or seed parent to conduct allogamous crossing, whereby 15 individuals of white color flowers, which was probability more than half. Blue color flower and purple color flower were used as pollen parent or seed parent to conduct allogamous crossing, whereby 12 individuals of purple color flowers, one individual of blue color, and one individual of bicolor (bicolor blue). Purple color flower and pale purple color flower were used as pollen parent or seed parent to conduct, whereby 9 individuals of purple color flowers. Pale purple color flower and white color flower were used as pollen parent or seed parent to conduct allogamous crossing, whereby 10 individuals of pale purple color flowers.

As shown in Table 2, light color flower and pale purple color flower were used as pollen parent or seed parent to conduct allogamous crossing, whereby 1 individual of bicolor blue, 7 individuals of bicolor light blue, and one individual of white flower were obtained. Light blue color flower and white color flower were used as pollen parent or seed parent to conduct allogamous crossing, whereby 4 individuals of bicolor light blue, and 4 individuals of light blue color flowers were obtained. From this simplified chart (Table 2), flower colors separated into progenies can be quickly understood

### Example 3

Blue Springs was used as pollen parent or seed parent to conduct allogamous crossing to obtain F₁ seeds. The F₁ seeds were cultivated on the laboratory dish under the condition they were germinated at approximately 15° C., the seedlings were cultivated, were flowering, and their progenies were examined. The results are shown in Table 3.

TABLE 3

| | | Blue Springs | | | | | |
|---|---|---|---|---|---|---|---|
| | | Blue | L. Blue | Purple | P. Purple | Red Pink | Pink |
| Blue Springs | Blue | B 4(44) | | | | | |
| | | Lb 5(55) | | | | | |
| | L. Blue | B 2(33) | PB 3(75) | | | | |
| | | Bb 6(60) | Pink 1 | | | | |
| | | Blb 2(20) | (25) | | | | |
| | Purple | B 2(20) | LB 1 | | | | |
| | | Bb 6(60) | (17) | | | | |
| | | Blb 1(17) | P 2(38) | | | | |
| | | | Blb 2(33) | | | | |
| | | | Bb 1(17) | | | | |
| | Pale | LB 7(30) | PP 9(41) | P 6(66) | PP 1(100) | | |
| | Purple | B 5(22) | Blb 13(59) | Bb 1(14) | | | |
| | | P 1(4) | | | | | |
| | | Blb 8(35) | | | | | |
| | | Bb 2(9) | | | | | |
| | Pink | PP 1(12) | P 4(44) | PP 3(43) | P 11(78) | Pink 1(100) | |
| | | Bb 6(75) | Bb 4(44) | P 2(28) | PP 2(14) | | |
| | | Blb 1(12) | Blb | Pink | Bb 1(7) | | |
| | | | 1(11) | 1(14) | | | |
| | | | | Blb 1(14) | | | |
| | Red | B 3(8) | Bb 5(100) | P 4(44) | P 8(80) | Red Pink | Red Pink |
| | Pink | P 1(3) | | Pink | Bb 1(10) | 5(100) | 5(55) |
| | | Pink 1(3) | | 1(11) | Blb 1(10) | | LB 1(11) |
| | | Bb 33(87) | | Red Pink | | | Bb 2(22) |
| | | | | 2(22) | | | Blb 1(11) |
| | | | | W 2(22) | | | |
| | White | LB 1(8) | W 11(34) | LB 1(33) | | W 1(12) | W 5(62) |
| | | W 1(8) | | Bb 1(33) | | Blb 5(62) | P 1(12) |
| | | Blb 8(61) | PP 3(9) | Blb 1(33) | | Bb 1(25) | Bb 2(25) |
| | | Bb 3(23) | | | | | |

B: Blue,
LB: Light Blue
P: Purple
PP: Pale Purple
W: White
Bb: B Blue
Blb: B Light Blue

As shown in Table 3, blue color flower and pale purple color flower were used as pollen parent or seed parent to conduct allogamous crossing, whereby 5 individuals of blue color flowers and 7 individuals of light blue color flowers, one individual of purple color flower, 2 individuals of bicolor blue, and 8 individuals of bicolor light blue were obtained. Light blue color flower and white color flower were used to conduct allogamous crossing, whereby 11 individuals of white color flowers and 3 individuals of pale purple color flowers, one individual of light blue color flower, and 17 individuals of bicolor light blue were obtained. Purple color flower and pale purple color flower were used as pollen parent or seed parent to conduct allogamous crossing, whereby 6 individuals of purple color flowers and one individual of bicolor blue were obtained. Pale purple color flower and pink color flower were used to conduct allogamous crossing, whereby 11 individuals of purple color flowers, 2 individuals of pale purple flowers and one individual of bicolor blue were obtained. Pink color flower and red pink color flower were used to conduct allogamous crossing, whereby 5 individuals of red pink flowers were obtained.

As shown in Table 3, blue color flower and red pink color flower were used as pollen parent or seed parent to conduct allogamous crossing, whereby mainly 33 individuals of bicolor blue flowers were obtained. Light blue color flower and pale purple flower were used to conduct allogamous crossing, whereby 9 individuals of pale purple color flowers and 13 individuals of bicolor light blue flowers were obtained. Light blue color flower and red pink color flowers were used to conduct allogamous crossing, whereby 5 individuals of bicolor blue flowers were obtained. Blue color flower and purple color flowers were used to conduct allogamous crossing, whereby 2 individuals of blue color flowers, 6 individuals of bicolor blue flowers, 2 individuals of bicolor light blue flowers were obtained. Blue color flower and white color flower were used to conduct allogamous crossing, whereby one individual of light blue color flower, one individual of white color flower, 3 individuals of bicolor blue flowers, and 8 individuals of bicolor light blue flowers were obtained. From this simplified chart (Table 2), flower colors separated into progenies can be quickly understood

### Example 4

Pacific Giant and Blue Springs were used as pollen parent or seed parent to conduct allogamous crossing to obtain F₁ seeds. The F₁ seeds were cultivated on the laboratory dish under the condition they were germinated at approximately 15° C., the seedlings were cultivated, were flowering, and their progenies were examined. The results are shown in Table 4.

TABLE 4

| | | Pacific Giant | | | | |
|---|---|---|---|---|---|---|
| | | Blue | L. Blue | Purple | P. Purple | White |
| Blue Springs | Blue | | | | | B 2(23) |
| | | | | | | LB 1(17) |
| | | | | | | Bb 2(33) |
| | | | | | | Blb 1(17) |
| | L. Blue | P 6(86) | LB 2(100) | | | PP 3(37) |
| | | B 1(14) | | | | Blb 1(14) |
| | Pink | B 1(33) | Blb 4(100) | P 5(71) | P 2(28) | PP 3(37) |
| | | Bb 2(66) | | PP 1(14) | PP 1(14) | Blb 3(37) |
| | | | | Bb 1(14) | Blb 3(43) | Bb 2(25) |
| | | | | | Bb 1(14) | |
| | Red Pink | B 2(12) | B 2(5) | P 4(44) | P 6(86) | W 8(100) |
| | | PP 1(6) | Bb 35(94) | Red Pink | Bb 1(14) | |
| | | W1(6) | | 3(33) | | |
| | | Bb 11(69) | | Bb 2(22) | | |
| | | Blb 1(6) | | | | |
| | White | W 1(100) | | P 6(60) | | |
| | | | | Lb 2(20) | | |
| | | | | PP 1(10) | | |
| | | | | Bb 1(10) | | |

B: Blue,
LB: Light Blue
P: Purple
PP: Pale Purple
W: White
Bb: B Blue
Blb: B Light Blue

As shown in Table 4, when Pacific Giant had light blue color flower and Blue Springs had red pink flower, mainly 35 individuals of bicolor blue flowers were obtained. From this simplified chart (Table 4), flower colors separated into progenies can be quickly understood

### Example 5

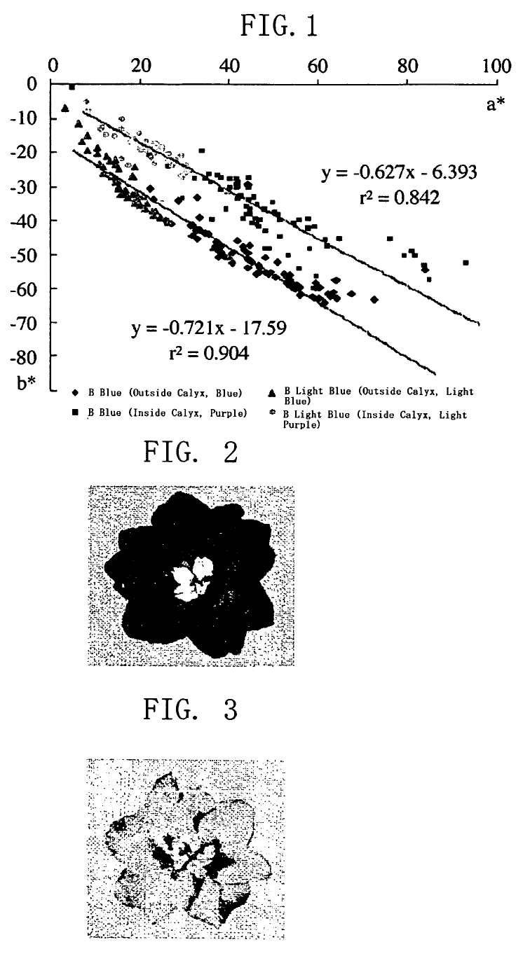

Flower colors of blue color type bicolor flower (bicolor blue, B blue) and light blue color type bicolor flower (bicolor light blue, B light blue) were measured according methods described in documents (Non-Patent Documents 11 and 12). The results are shown in FIG. 1. Photos of blue color type bicolor flower (bicolor blue, B blue) and light blue color type bicolor flower (bicolor light blue, B light blue) are shown in FIG. 2 and FIG. 3, respectively. It can be understood that inside and outside of calyx were clearly different from each other. Flower color of inside of the calyx were distributed from purple to pale purple for blue color type bicolor flower (bicolor blue, B blue) and light blue color type bicolor flower (bicolor light blue, B light blue), and according to tropics equation (y=−0.627x−6.393), the correlation coefficient was 0.842, and thus, it was understood to have significant relation. Flower color of outside of the calyx were distributed from blue purple to blue on the figure for blue color type bicolor flower (bicolor blue, B blue) and light blue color type bicolor flower (bicolor light blue, B light blue), and according to tropics equation (y=−0.721x−17.59), the correlation coefficient was 0.904, and thus, it was understood to have significant relation.

### Example 6

Pigment compositions of blue color type bicolor flower (bicolor blue, B blue) and light blue color type bicolor flower (bicolor light blue, B light blue) were measured according methods described in documents (Non-Patent Documents 11 and 12). The results are shown in Table 5.

TABLE 5

| | Intrinsic Pigment | | | | | | |
|---|---|---|---|---|---|---|---|
| | (nano mol/fresh weight g) | | | | | | |
| Delphinium Bicolor | 1 | 2 | 3 | A | B | C | 4 |
| B Blue (Outside Calyx blue) | 0.3 | 0.8 | 82.2 | 6.1 | 6.5 | 3.5 | 120.7 |
| B Blue (Inside Calyx Purple) | 0.4 | 0.8 | 88.5 | 7.3 | 10.2 | 3.4 | 126.0 |
| B Light Blue (Outside Calyx | 0.0 | 0.1 | 7.8 | 0.5 | 2.8 | 0.3 | 38.4 |
| Light Blue) | | | | | | | |
| B Light Blue (Inside Calyx | 0.0 | 0.1 | 10.2 | 0.6 | 2.6 | 0.2 | 44.2 |
| Light Blue) | | | | | | | |

It can be understood from Table 5 that the pigment composition of the inside and outside of the calyx of bicolor are similar. As intrinsic pigment in Table 5, 1 stands for bisdeacylplatyconin, 2 stands for tulipanin, 3 stands for violdelphin, and 4 stands for cyanodelphin. Although c is pigment having not yet been identified in Table 5 as an intrinsic pigment, separation through high speed liquid chromatography can be done in a well manner. Pigments a and b are a novel anthocyanin represented by formula (I) and an anthocyanin represented by formula (II), respectively. Retention time of each of pigments were 9.1 minutes for 1, 15.9 minutes for 2, 31.6 minutes for 3, 34.2 minutes for pigment a (I), 34.7 minutes for pigment b (II), 51.6 minutes for c, and 58.5 minutes for 4.

### Example 7

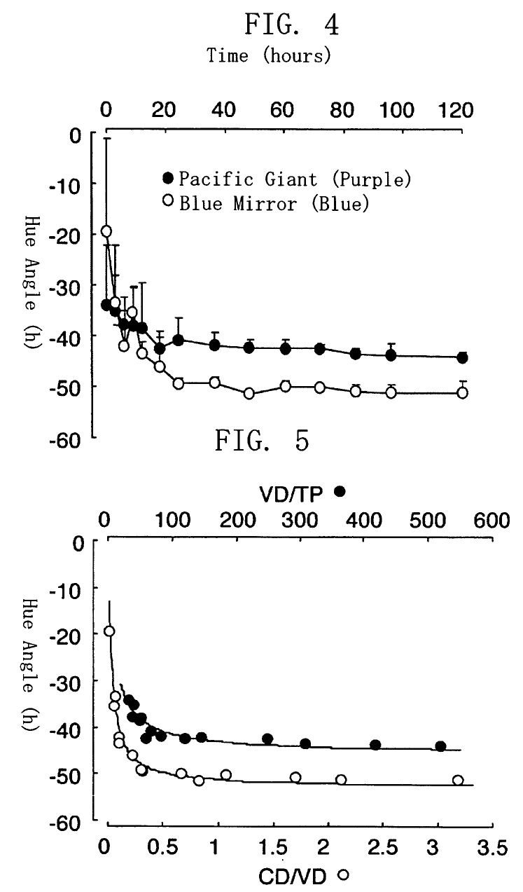

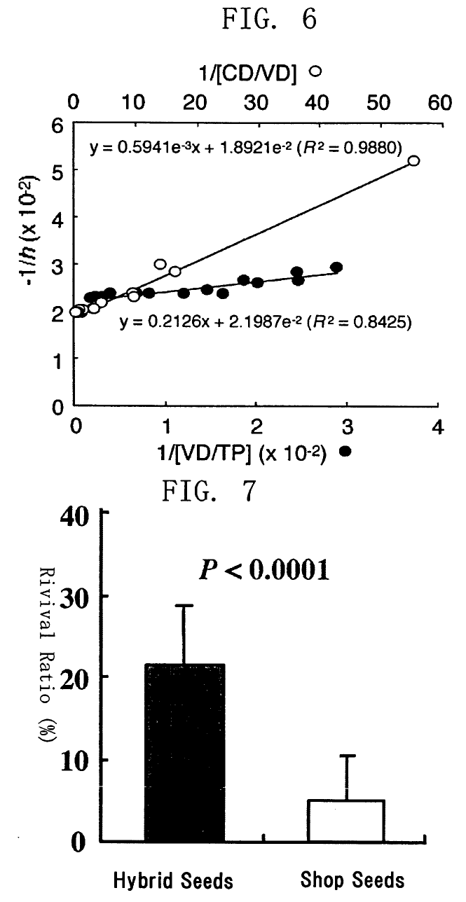

The flower color after flowering and the intrinsic pigment of calyx were examined. The periods of the examination were immediately after flowering, 3, 6, 9, 12, 18, 24, 36, 48, 60, 72, 84, 96, and 120 hours after flowering. FIG. 4 shows the change in hue angle (h) of delphinium Pacific Giant (purple color) and Blue Mirror (blue) with the elapse of time. As for Pacific Giant (Purple), the ratio of tulipanin (TP) to violdelphin (VD) which were main intrinsic pigment of the calyx was taken as the horizontal axis, and as for Blue Mirror (blue), the ratio of violdelphin, (VD) to cyanodelphin (CD) which were main intrinsic pigment of the calyx was taken as the horizontal axis. The hue angles were taken as the vertical axis, and their relations are shown in FIG. 5. FIG. 6 shows the reciprocal values those plotted on FIG. 5.

From FIG. 6, equation 1 and equation 2 were lead. From these results, it has been found that the ratio of major intrinsic pigment and flower color have the relation shown in equation 1 and equation 2.

$$H = {H}_{max} \frac{[ CD / VD ]}{[ CD / VD ] + {K}_{H}} = {tang}^{- 1} ( {b}^{*} / {a}^{*} ) \qquad ( 1 )$$

wherein CD/VD exhibits ratio of major intrinsic pigment within the calyx of delphinium; CD is Cyanodelphin, VD is of Violdelphin, Hmax is the maximum hue angle showing flower color, and K_{H} is a constant of ratio of intrinsic pigment in the case of half of the maximum, hue angle.

$$\frac{1}{H} = \frac{1}{{H}_{max}} + \frac{{K}_{H}}{{H}_{max}} \cdot \frac{1}{[ CD / VD ]} \qquad ( 2 )$$

Based on the relation of formula 1 and relation of formula 2, the flower color of Blue Mirror (blue), Pacific Giant (blue), Pacific Giant (light blue), Pacific Giant (purple), Pacific Giant (pale purple) after flowering, and the intrinsic pigment of calyx were examined. The examination periods were immediately after flowering, 3, 6, 9, 12, 18, 24, 36, 48, 60, 72, 84, 96, and 120 hours after flowering, and the maximum hue angle (Hmax) and K_{H} were calculated. The results are shown in Table 6.

TABLE 6

| | Flower | Intrinsic | | |
|---|---|---|---|---|
| Delphinium | Color | Pigment | Hmax (°) | KH |
| Blue Mirror | Blue | CD/VD | −51.3 | 0.01 |
| Pacific Giant | Blue | CD/VD | −51.9 | 0.06 |
| Pacific Giant | Light Blue | CD/VD | −72.8 | 0.37 |
| Pacific Giant | Purple | VD/TP | −45.3 | 6.60 |
| Pacific Giant | Pale Purple | VD/TP | −55.3 | 7.23 |

From Table 6, the maximum hue angle (Hmax), i.e., matured inflorescence can be known from the intrinsic pigment of calyx.

### Example 8

pH value of calyx after flowering was examined. The examination periods were immediately after flowering and 3, 6, 9, 12, 18, 24, 36, 48, 60, 72, 84, 96, and 120 hours after flowering. The results are shown in Table 7.

TABLE 7

| Del. | Time After Flowering (hr) | | | | | | | | | | | | | |
|---|---|---|---|---|---|---|---|---|---|---|---|---|---|---|
| BM | 0 | 3 | 6 | 9 | 12 | 18 | 24 | 36 | 48 | 60 | 72 | 84 | 96 | 120 |
| PG | 5.22 | 5.30 | 5.31 | 5.24 | — | 5.31 | 5.29 | 5.22 | 5.23 | 5.20 | 5.35 | 5.18 | 5.30 | 5.29 |
| PG | 5.14 | 5.10 | 5.03 | 5.19 | 5.22 | 5.22 | 5.24 | 5.27 | 5.26 | 5.21 | 5.28 | 5.21 | 5.28 | 5.33 |
| PG | 5.03 | 5.08 | 5.04 | 5.17 | 5.11 | 5.07 | 5.13 | 5.16 | 5.17 | 5.15 | 5.19 | 5.16 | 5.18 | 5.19 |
| PG | 5.20 | 5.15 | 5.23 | 5.08 | 5.18 | 5.17 | 5.36 | 5.21 | 5.28 | 5.35 | 5.38 | 5.46 | 5.46 | 5.43 |
| PG | 5.25 | 5.31 | 5.24 | 5.26 | 5.32 | 5.25 | 5.24 | 5.35 | 5.27 | 5.26 | 5.25 | 5.35 | 5.36 | 5.38 |

BM Blue Mirror
PG Pacific Giant

From Table 7, it has been proven that pH value is not changed.

### Example 9

### Comparison of Cultivation Method for Raising Seedling by Germinating Self-Proliferated Seeds Within a Laboratory Dish at 15° C. with Cultivation Method for Raising Seedlings by Directly Seeding at June

Commercially available Beradonna Beranotherm, Beradonna Casablanca, Blue Mirror, Pacific Giant, Blue Springs, Magic Founten seeds are self-propagated at spring of 1999, to obtain seeds. The self-propagated seeds were seeded within a laboratory dish at the middle of August 1999.

At the middle of August 2000, self-propagated seeds were seeded within a laboratory dish. Defatted cotton were previously placed within the laboratory dish, and water was absorbed thereon in such an seeds were half sunk within the defatted cotton. The laboratory dish within which seeds were seeded was placed within a refrigerator at 15° C. for about 7 days to 10 days under dark conditions to germinate seeds. The germinated seeds were planted on a cell tray one after another. The planted cell tray was cultivated within a greenhouse at a high temperature of from 25 to 32° C., and seedlings were transferred to vinyl house. For promoting growth, heating through a heater was started from the last ten days of December to keep the temperature of the vinyl house at approximately 15° C. In order to accelerate differentiation of flower bud, long day treatment with lighting was conducted from the middle ten day of January 2001 to the last ten days of April 2001. Lighting conditions were as follows: Height from ridge was 1.1 m, one 100 W incandescent lamp per an area of 9 m², lighting over a period of 6 hours from 9:00 p.m. to 3:00 a.m. Number of seeds seeded within the laboratory dish was counted, and number of delphinium which was flowering from April to May, 2001 was counted to calculate revival (flowering) ratio of the direct seedlings.

TABLE 8

| | Revival (Flowering) Ratio | | |
|---|---|---|---|
| | (%) in Each year | | |
| Kind of Delphinium | 2000 | 2001 | 2002 |
| Directly Seeded at June of Previous | | | |
| Year | | | |
| Beradonna Beranotherm (Blue) | 4.0 | 6.0 | 0 |
| Beradonna Casablanca (White) | 0 | 5.0 | 0 |
| Blue Mirror (Blue) | 0 | 21.3 | 10.2 |
| Pacific Giant | 2.0 | 2.6 | 0 |
| Blue Springs | 0 | 4.0 | — |
| Magic Fountain | 0 | 0 | 0 |
| Clear Springs | 0.5 | 1.7 | — |
| Seeded Within Lab. Dish at August of | | | |
| Previous Year | | | |
| Beradonna Beranotherm (Blue) | 33.2 | 8.3 | 2.2 |
| Beradonna Casablanca (White) | 18.2 | — | 25.3 |
| Blue Mirror (Blue) | 19.8 | — | 25.3 |
| Pacific Giant | 14.3 | 24.5 | — |
| Blue Springs | 3.6 | 11.4 | 1.1 |
| Magic Fountain | 7.9 | 2.8 | 3.3 |
| Clear Springs | 21.9 | 1.6 | — |

As a result of Table 8, as for raising seedlings and cultivation of various delphiniums, revival ratio of those seeded within the laboratory dish are higher.

### Example 10

Comparison of revival ratio between hybrid seeds and purchased seeds was conducted. As hybrid seeds, 9 lines of allogamic crossed Pacific Giant were used. Both were cultivated by seeding the seeds within a laboratory dish followed by raising seedlings, and the revival (flowering) ratio was calculated. The results are shown in FIG. 7. As for the hybrid seeds, the ratio was shown as an average and standard deviation of 9 lines, and as for purchased seeds, the ratio was shown as an average and standard deviation of 5 lines. As a result, whereas the hybrid seeds showed the revival ratio of approximately 22%, the purchased seeds showed up to approximately 5%, indicating that hybrid seeds are more vital (FIG. 7).

### Example 11

### Method for Extracting Pigments (I) and (II) from Calyx of Blue Delphinium, and Isolating and Purifying the Same

Calyx (flower) of Blue delphinium (blue, light blue, purple and pale purple color delphinium) was collected. The weight of calyx was 11.6 kg. A solution (50% acetic acid/methanol) composed of 1:1 mixture of acetic acid with ethanol was added thereto to extract anthocyanin pigment. The extracted solution was filtrated through a cotton plug, and then the solvent was distilled out under a reduced pressure by a rotary evaporator. The extracted residue is dissolved in an aqueous 5% acetic acid solution, and was subjected to an open column chromatography. The conditions of the open column chromatography were as follows: As stationary layer, MCI gel CHP-20P (CHP-20P, Mitsubishi Chemical Corporation), Sephadex LH-20 (Pharmacia Biotech), (Chromatorex ODS, Fuji Silysia Chemical LTD.) are used. As mobile layer, an aqueous 5% acetic acid solution as A liquid and an aqueous 5% acetic acid-methanol solution as B liquid were used. By increasing the contents from A liquid to B liquid, various chromatographic tests are conducted. As a stationary phase, Sephadex LH-20 (Pharmacia Fine Chemical) was used, as for mobile phase, an aqueous 5% acetic acid solution is used as A liquid, and an aqueous 5% acetic acid-acetone solution is used as C liquid. By increasing the contents from C liquid to A liquid, various chromatographic tests are conducted. By repeating these open column chromatographic tests, it has been found that novel anthocyanin pigment represented by formula (I) (29.9 mg) and anthocyanin pigment represented by formula (II) (62.4 mg) were isolated. They were purple powder. In addition, monodeacylcampanin (32.8 mg) was also isolated which is a known pigment isolated from Campanula and whose structure has been decided (Brandt, K., Phytochem. 33:209-212, 1993). We made it clear for the first time to the fact that monodeacylcampanin is contained in calyx of delphinium.

When raising seedling under the conditions that the delphinium described above is germinated within a laboratory dish at a temperature of 15° C.±1° C. delphinium can be effectively growing (at a season), and particularly at a warm place.

### Example 12

The results of ¹H-Nuclear magnetic resonance [NMR] spectrum of novel anthocyanin (29.9 mg) represented by formula (I) are as follows:

¹H-NMR (500 MHz, CD₃OD+CF₃COOD, 9:1) d: 1.26 (3H, d, J=6.1 Hz, rha-6-CH₃), 3.30-4.00 (sugar-H), 4.02 (1H, br t, J=9.8 Hz, glc[II]-H-6b), 4.16 (1H, br d, J=10.9 Hz, 3-O-glc-H-6a), 4.29 (1H, brt, J=9.1 Hz, glc[I]-H-6b), 4.46 (1H, d, J=7.3 Hz, glc[II]-H-1), 4.63 (1H, br d, J=11.6 Hz, glc[II]-H-6a), 4.69 (1H, d, J=7.3 Hz, glc[III]-H-1), 4.82 (1H, s, rha-H-1), 5.02 (1H, br d, J=10.9 Hz, glc[I]-H-6a), 5.34 (1H, d, J=7.3 Hz, 3-O-glc-H-1), 5.41 (1H, d, J=7.3 Hz, glc[I]-H-1), 6.50 (2H, d, J=8.5 Hz, p-HBA[II]-H-3′,5′), 6.70 (1H, s, H-6), 6.82 (2H, d, J=8.5 Hz, p-HBA[I]-H-3′,5′), 7.10 (1H, s, H-8), 7.44 (2H, d, J=8.5 Hz, p-HBA[II]-H-2′,6′), 7.79 (2H, s, H-2′,6′), 7.84 (2H, d, J=8.5 Hz, p-HBA[I]-H-2′,6′), 8.66 (1H, s, H-4).

The results of ¹³C-Nuclear magnetic resonance [NMR] spectrum of novel anthocyanin (29.9 mg) represented by formula (I) are as follows:

¹³C-NMR (500 MHz, CD₃OD+CF₃COOD, 9:1) d: 17.9 (rha-C-6), 62.6 (glc[III]-C-6), 65.6 (glc[II]-C-6), 65.9 (glc[I]-C-6), 67.9 (3-O-glc-C-6), 69.7 (rha-C-5), 72.6 (rha-C-2), 70.8, 71.3, 71.6, 71.8, 72.5, 73.6, 74.1, 74.4, 74.5, 74.9, 75.5, 75.7, 77.8, 77.9, 78.0, 78.1, 78.2, 87.3 (glc[I]-C-3), 95.0 (C-8), 100.4 (glc[I]-C-1), 100.8 (glc[II]-C-1), 102.0 (rha-C-1), 103.4 (3-O-glc-C-1), 104.6 (C-6), 105.2 (glc[III]-C-1), 113.7 (C-10), 113.8 (C-2′,6′), 116.1 (p-HBA[II]-C-3′,5′), 116.3 (C-1′), 117.3 (p-HBA[I]C-3′,5′), 121.7 (p-HBA[II]-C-1′), 124.8 (p-HBA[I]-C-1′), 132.9 (p-HBA[I]-C-2′,6′), 133.0 (p-HBA[II]-C-2′,6′), 133.8 (C-4), 146.7 (C-4′), 147.1 (C-3), 147.6 (C-3′,5′), 156.1 (C-9), 158.0 (C-5), 162.3 (p-HBA[I]-C-4′), 163.1 (p-HBA[II]-C-4′), 164.1 (C-2), 166.9 (C-7), 167.4 (p-HBA[I]-COO), 167.5 (p-HBA[II]-COO).

The weight of novel anthocyanin (29.9 mg) represented by formula (I) was determined by high resolution mass spectrography (position-ion HR FAB-MS). The theoretic value was C₅₉H₆₉O₃₅: 1337.3620, and measured value was m/z: 1337.3934 [M]⁺, both are in agreement with each other.

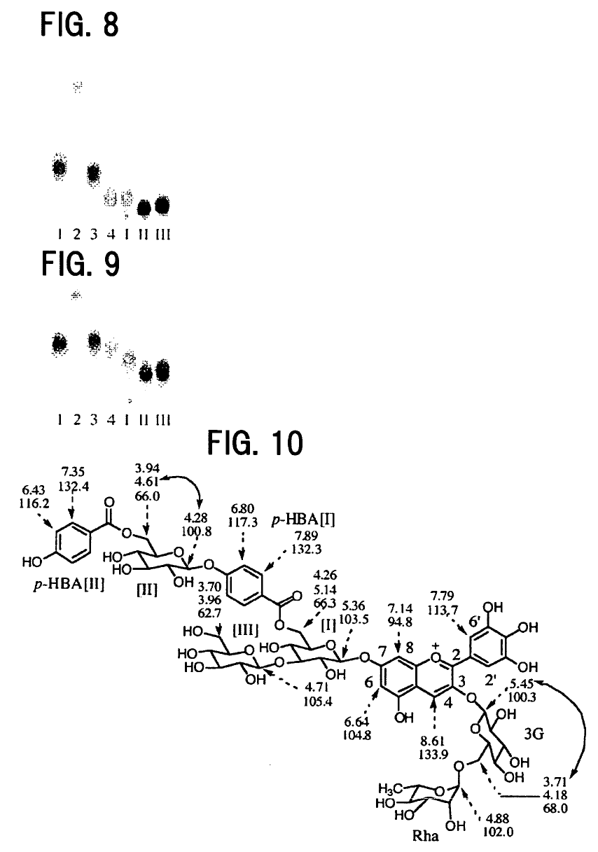

FG-HMQC, (field gradient-heteronuclear multiple quantum coherence spectrum) and ¹H-¹H TOCOSY, ¹H-¹H total correlation spectroscopy were measured. The results are shown in FIG. 8. Numbers in Figure is contribution of 13 Carbon (¹³C) relative to proton (¹H). Those having arrows on both sides of curve indicate the correlation of ¹H-¹H TOCOSY.

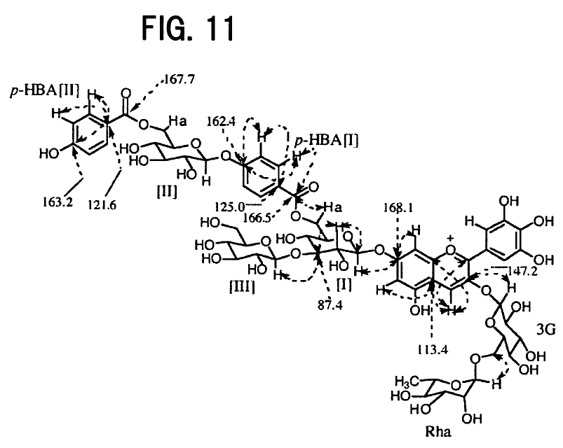

FG-HMBC, field gradient-heteronuclear multiple bond coherence spectrum of 13 Carbon (¹³C) relative to proton (¹H) was measured by NMR spectrum. The results are shown in FIG. 9. From these results, it can be understood that the chemical structure of the novel anthocyanin pigment can be represented by formula (I).

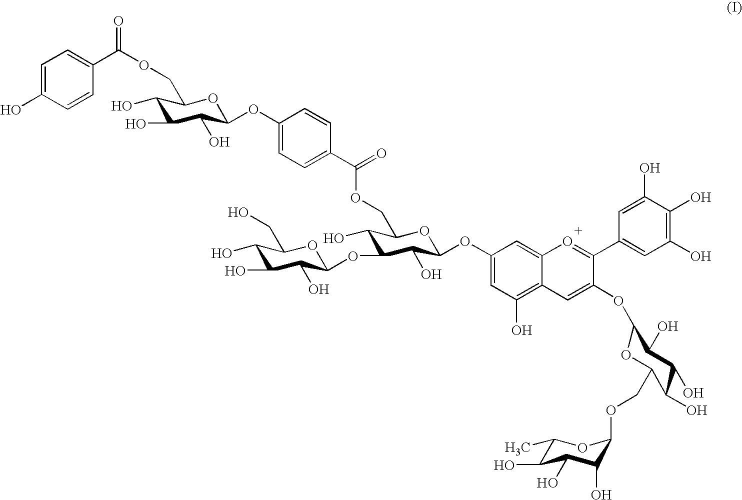

### Example 13

The results of ¹H-Nuclear magnetic resonance [NMR] spectrum of anthocyanidin (62.4 mg) represented by formula (II) are as follows:

H-NMR (500 MHz, CD₃OD+CF₃COOD, 9:1) d: 1.26 (3H, d, J=5.5 Hz, rha-6-CH₃), 3.30-4.00 (sugar-H), 4.03 (1H, m, glc[II]-H-6b), 4.17 (1H, br d, J=10.3 Hz, 3-O-glc-H-6a), 4.28 (1H, m, glc[I]-H-6b), 4.46 (1H, br s, glc[II]-H-1), 4.61 (1H, br s, glc[III]-H-1), 4.62 (1H, m, glc[II]-H-6a), 4.77 (1H, br s, glc[IV]-H-1), 4.82 (1H, s, rha-H-1), 5.02 (overlapped, glc[I]-H-6a), 5.35 (1H, br s, 3-O-glc-H-1), 5.41 (1H, br s, glc[I]-H-1), 6.49 (2H, d, J=7.9 Hz, p-HBA[II]-H-3′,5′), 6.69 (1H, s, H-6), 6.81 (2H, d, J=7.3 Hz, p-HBA[I]-H-3′,5′), 7.09 (1H, s, H-8), 7.43 (2H, d, J=7.9 Hz, p-HBA[II]-H-2′,6′), 7.77 (2H, s, H-2′,6′), 7.83 (2H, d, J=7.3 Hz, p-HBA[I]-H-2′,6′), 8.65 (1H, s, H-4).

The results of ¹³C-Nuclear magnetic resonance [NMR] spectrum of anthocyanidin (62.4 mg) represented by formula (II) are as follows:

¹³C-NMR (500 MHz, CD₃OD+CF₃COOD, 9:1) d: 17.9 (rha-C-6), 62.1, 62.6 (glc[III,IV]-C-6), 65.6 (glc[II]-C-6), 65.9 (glc[I]-C-6), 67.9 (3-O-glc-C-6), 69.7 (rha-C-5), 70.0, 70.6, 71.3 ,71.6, 71.8, 72.5, 72.6, 73.3, 73.7, 74.0, 74.4, 74.5, 74.8, 75.5 (×2), 75.7, 77.8 (×2), 77.9 (×2), 78.1, 87.1 (glc[III]-C-3), 87.4 (glc[I]-C-3), 95.0 (C-8), 100.4 (glc[I]-C-1), 100.8 (glc[II]-C-1), 101.9 (rha-C-1,glc[IV]-C-1), 103.4 (3-O-glc-C-1), 104.8 (C-6), 105.2 (glc[III]-C-1), 113.8 (C-10,C-2′6′), 116.1 (p-HBA(II]-C-3′,5′), 116.3 (C-1′), 117.2 (p-HBA[I]-C-3′,5′), 121.6 (p-HBA[II]-C-1′), 124.8 (p-HBA[I]-C-1′), 132.1 (p-HBA[II]-C-2′,6′), 132.3 (p-HBA[I]-C-2′,6′), 133.8 (C-4), 146.7 (C-4′), 147.0 (C-3), 147.6 (C-3′,5′), 156.1 (C-9), 158.0 (C-5), 162.2 (p-HBA[II]-C-4′), 163.0 (p-HBA[I]-C-4′), 164.1 (C-2), 166.9 (C-7), 167.4 (p-HBA[II]-COO), 167.5 (p-HBA[I]-COO).

The weight of anthocyanidin (62.4 mg) represented by formula (II) was determined by high resolution mass spectrography (position-ion HR FAB-MS). The theoretic value was C₆₅H₇₉O₄₀: 1499.4148, and measured value was m/z: 1499.4281 [M]⁺, both are in agreement with each other.

From these results, the chemical structure of the anthocyanin pigment represented by formula (II) has been found to be the same as that of bisdeacylcyanodelphin obtained by hydrolyzing cyanodelphin portion described in Non-Patent Document 7. It can be understood from this result for the first time that the anthocyanin pigment represented by formula (II) is not contained as any derivative or synthesized product, but is contained in calyx of delphinium as a natural pigment.

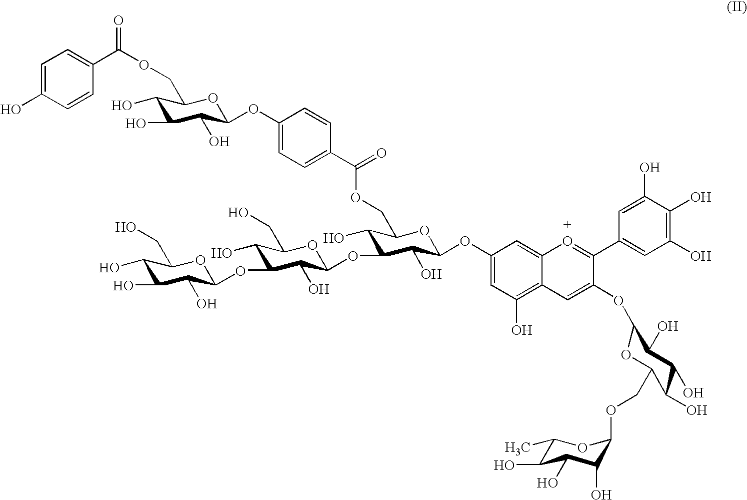

### Example 14

The results of ¹H-Nuclear magnetic resonance [NMR] spectrum of monodeacylcampanin (32.8 mg) being known antocyanine pigment, are as follows:

¹H-NMR (500 MHz, CD₃OD+CF₃COOD, 9:1) d: 1.30 (3H, d, J=6.1 Hz, rha-6-CH₃), 3.30-4.00 (sugar-H), 3.90 (1H, m, glc[II]-H-6b), 4.13 (1H, br t, J=10.9 Hz, glc[I]-H-5), 4.19 (1H, br d, J=11.4 Hz, 3-O-glc-H-6a), 4.23 (1H, d, J=6.7 Hz, glc[II]-H-1), 4.33 (1H, br t, J=10.4 Hz, glc[I]-H-6b), 4.65 (1H, br d, J=11.6 Hz, glc[II]-H-6a), 4.90 (1H, s, rha-H-1), 4.93 (1H, d, J=7.9 Hz, glc[III]-H-1), 4.99 (1H, br d, J=10.4 Hz, glc[I]-H-6a), 5.28 (1H, d, J=7.9 Hz, 3-O-glc-H-1), 5.37 (1H, d, J=7.9 Hz, glc[I]-H-1), 6.63 (2H, d, J=9.1 Hz, p-HBA[II]-H-3′,5′), 6.72 (1H, s, H-6), 6.90 (2H, d, J=8.5 Hz, p-HBA[I]-H-3′,5′), 7.21 (1H, s, H-8), 7.41 (2H, d, J=8.5 Hz, p-HBA[II]-H-2′,6′), 7.90 (2H, s, H-2′,6′), 7.99 (2H, d, J=9.1 Hz, p-HBA[I]-H-2′,6′), 8.55 (1H, s, H-4).

The results of ¹H-Nuclear magnetic resonance [NMR] spectrum of monodeacylcampanin (32.8 mg) being known antocyanine pigment, are as follows:

The results of ¹³C-Nuclear magnetic resonance [NMR] spectrum of monodeacylcampanin (32.8 mg) being known antocyanine pigment, are as follows:

¹³C-NMR (500 MHz, CD₃OD+CF₃COOD, 9:1) d: 17.9 (rha-C-6), 62.4 (glc[III]-C-6), 66.3 (glc[I]-C-6), 66.4 (glc[II]-C-6), 67.9 (3-O-glc-C-6), 69.8 (rha-C-5), 72.7 (rha-C-2), 74.3 (glc[II]-C-5), 76.1 (glc[I]-C-5), 71.3, 71.5, 71.8, 72.3, 72.8, 74.1, 74.2, 74.5, 74.6, 74.7, 77.6, 77.7 (×2), 78.0 (×2), 78.2 (rha-C-3,4,4×glc-C-2,3,4, glc[III]-C-5, 3-O-glc-C-5), 94.5 (C-8), 100.2 (glc[III]-C-1), 101.0 (glc[I,II]-C-1), 101.9 (rha-C-1), 103.8 (3-O-glc-C-1), 105.4 (C-6), 113.7 (C-10), 113.8 (C-2′6′), 116.3 (p-HBA[II]-C-3′,5′), 117.4 (p-HBA[I]-C-3′,5′), 119.5 (C-1′), 124.1 (p-HBA[II]-C-1′), 125.2 (p-HBA[I]-C-1′), 131.7 (p-HBA[II]-C-2′,6′), 132.1 (p-HBA[I]-C-2′,6′), 134.1 (C-4), 146.9 (C-4′), 147.3 (C-3), 147.9 (C-3′,5′), 156.3 (C-9), 157.8 (C-5), 162.0 (p-HBA[II]-C-4′), 162.5 (p-HBA[I]-C-4′), 163.9 (C-2), 166.9 (p-HBA[II]-COO), 167.1 (C-7), 167.5 (p-HBA[I]-COO).

The weight of monodeacylcampanin (32.8 mg) being known antocyanine pigment was determined by high resolution mass spectrography (position-ion HR FAB-MS) . The theoretic value was C₅₉H₆₉O₃₅: 1337.3620, and measured value was m/z: 1337.3732 [M]⁺, both are in agreement with each other.

From these results, the chemical structure of monodeacylcampanin (32.8 mg) being known anthocyanin pigment, isolated from the calyx of delphinium has been found to be the same as that of monodeacylcampanin described in literature (Brandt, K., Phytochem. 33:209-212, 1993). It can be understood from this result for the first time that monodeacylcampanin represented by formula (III) is not contained as any derivative or synthesized product, but is contained in calyx of delphinium as a natural pigment.

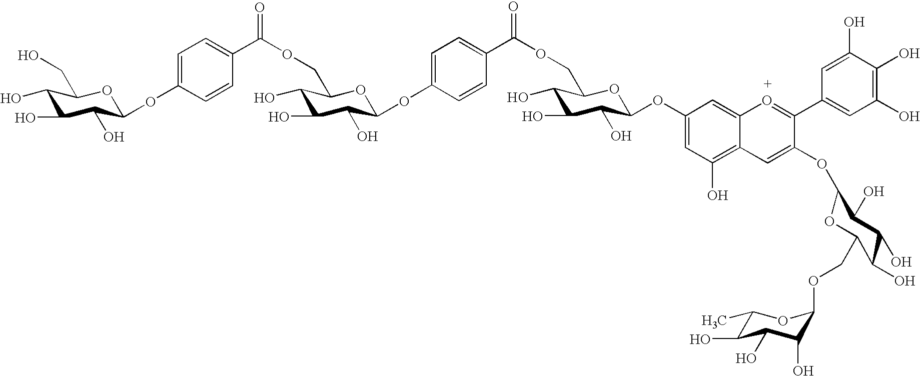

### Example 15

The self-propagated seeds described above were seeded within a laboratory dish, the seedlings was raising, and growing to obtain pale purple Pacific Giant. The calyx of the resulting pale purple Pacific Giant was examined for anthocyanin. The results are shown in Table 9. It can be understood that the delphinium contains the novel anthocyanin pigment (I), and also contains anthocyanin pigment (II) as a main pigment. Up to now, purple color delphinium has been said to contain violdelphin as dominant pigment, but, it has been understood from these results that some individuals contain anthocyanin pigment (II) content of which is higher than that of violdelphin. The anthocyanin pigment shown by (III) in Table 9 is a concentration of monodeacylcampanin. As intrinsic pigment in Table 9, 1 stands for bisdeacylplatyconin, 2 stands for tulipanin), 3 stands for violdelphin, and 4 stands for cyanodelphin.

TABLE 9

| | CIELab | | | Intrinsic Pigment (nano mol/fresh weight g) | | | | | | |
|---|---|---|---|---|---|---|---|---|---|---|
| Pacific Giant | L* | C* | H | 1 | 2 | (III) | 3 | (I) | (II) | 4 |
| Pale Purple Flower | 58.7 | 31.1 | −46.2 | 0.0 | 0.0 | 43.9 | 340.5 | 42.1 | 428.6 | 0.0 |

### Example 16

Blue color Pacific and pale purple color flower Pacific Giant were subjected to allogamous crossing, the seeds were seeded within the above-mentioned laboratory dish, the seedlings were raising and growing to obtain hybrid Pacific Giant having purple color flower and pale purple color flower whose calyx was examined for anthocyanin. The results are shown in Table 10. Purple color flower Pacific Giant and pale purple color flower Pacific Giant each containing the novel anthocyanin (I) and also containing anthocyanin pigment (II) as a major pigment can be found to be obtained by the crossing. Also, the flower color and anthocyanin within the calyx were examined for commercially available seeds of purple flower Pacific Giant. As a result, the purple flower Pacific Giant obtained by allogamous crossing (8 individuals) had more bright flower color and somewhat bluish in comparison with the commercially available seeds of purple flower (Table 10). The anthocyanin pigment shown by (III) in Table 10 is a concentration of monodeacylcampanin. As intrinsic pigment in Table 10, 1 stands for bisdeacylplatyconin, 2 stands for tulipanin), 3 stands for violdelphin, and 4 stands for cyanodelphin.

TABLE 10

| | | CIELab | | | Intrinsic Pigment (nano mol/fresh weight g) | | | | | | |
|---|---|---|---|---|---|---|---|---|---|---|---|
| Pacific Giant | Ind. | L* | C* | h | 1 | 2 | (III) | 3 | (I) | (II) | 4 |

Crossing Parent (Reciprocal Crossing)

| | | | | | | | | | | | |
|---|---|---|---|---|---|---|---|---|---|---|---|
| Blue | 1 | 37.3 | 71.6 | −51.9 | 16.6 | 5.2 | 49.2 | 703.7 | 72.3 | 116.1 | 881.0 |
| Pale Purple | 1 | 58.7 | 31.1 | −46.2 | 0.0 | 0.0 | 43.9 | 340.5 | 42.1 | 428.6 | 0.0 |

Allogamous Crossing

| | | | | | | | | | | | |
|---|---|---|---|---|---|---|---|---|---|---|---|
| Purple | 8 | 46.9 | 52.7 | −46.8 | 0.0 | 2.0 | 53.7 | 243.5 | 32.3 | 768.1 | 0.0 |
| Pale Purple | 3 | 50.8 | 48.2 | −46.3 | 0.0 | 0.0 | 44.9 | 165.9 | 32.4 | 364.2 | 0.0 |

Commercially Available Seeds

| | | | | | | | | | | | |
|---|---|---|---|---|---|---|---|---|---|---|---|
| Purple | 1 | 26.8 | 78.7 | −37.2 | 72.3 | 3.4 | 91.3 | 10980 | 113.2 | 22.5 | 0.0 |

### Example 17

Blue color Pacific and white color flower Pacific Giant were subjected to allogamous crossing, the seeds were seeded within the above-mentioned laboratory dish, the seedlings were raising and growing to obtain hybrid Pacific Giant having purple color flower whose calyx was examined for anthocyanin. The results are shown in Table 11. Purple color flower Pacific Giant containing the novel anthocyanin (I) and also containing anthocyanin pigment (II) as a major pigment can be found to be obtained by the crossing. The anthocyanin pigment shown by (III) in Table 11 is a concentration of monodeacylcampanin. As intrinsic pigment in Table 11, 1 stands for bisdeacylplatyconin, 2 stands for tulipanin), 3 stands for violdelphin, and 4 stands for cyanodelphin.

TABLE 11

| | | CIELab | | | Intrinsic Pigment (nano mol/fresh weight g) | | | | | | |
|---|---|---|---|---|---|---|---|---|---|---|---|
| Pacific Giant | Ind. | L* | C* | h | 1 | 2 | (III) | 3 | (I) | (II) | 4 |

Crossing Parent (Reciprocal Crossing)

| | | | | | | | | | | | |
|---|---|---|---|---|---|---|---|---|---|---|---|
| Blue | 2 | 38.9 | 72.6 | −50.6 | 8.3 | 2.6 | 62.9 | 580.4 | 52.9 | 67.2 | 1027.4 |
| White | 2 | 80.1 | 3.5 | −2.1 | 652.4 | 0.0 | 0.0 | 0.0 | 0.0 | 0.0 | 0.0 |

Allogamous Crossing

| | | | | | | | | | | | |
|---|---|---|---|---|---|---|---|---|---|---|---|
| Purple | 1 | 43.2 | 56.8 | −46.7 | 0.0 | 2.3 | 81.3 | 888.6 | 0.0 | 1008.3 | 0.0 |

Ind. Individual Number

### Example 18

Light blue color Pacific and white color flower Pacific Giant were subjected to allogamous crossing, the seeds were seeded within the above-mentioned laboratory dish, the seedlings were raising and growing to obtain hybrid Pacific Giant having purple color flower whose calyx was examined for anthocyanin. The results are shown in Table 12. Purple color flower Pacific Giant containing the novel anthocyanin (I) and also containing anthocyanin pigment (II) as a major pigment can be found to be obtained by the crossing. The anthocyanin pigment shown by (III) in Table 11 is a concentration of monodeacylcampanin. As intrinsic pigment in Table 12, 1 stands for bisdeacylplatyconin, 2 stands for tulipanin), 3 stands for violdelphin, and 4 stands for cyanodelphin.

TABLE 12

| | | CIELab | | | Intrinsic Pigment (nano mol/fresh weight g) | | | | | | |
|---|---|---|---|---|---|---|---|---|---|---|---|
| Pacific Giant | Ind. | L* | C* | h | 1 | 2 | (III) | 3 | (I) | (II) | 4 |

Crossing Parent (Reciprocal Crossing)

| | | | | | | | | | | | |
|---|---|---|---|---|---|---|---|---|---|---|---|
| Light Blue | 1 | 55.9 | 44.8 | −58.2 | 0.0 | 0.0 | 11.2 | 50.3 | 4.6 | 2.7 | 325.6 |
| White | 1 | 81.0 | 3.2 | 13.7 | 692.6 | 0.0 | 0.0 | 0.0 | 0.0 | 0.0 | 0.0 |

Allogamous Crossing

| | | | | | | | | | | | |
|---|---|---|---|---|---|---|---|---|---|---|---|
| Purple | 1 | 39.3 | 63.3 | −43.7 | 0.0 | 135.1 | 65.2 | 662.3 | 99.5 | 775.2 | 0.0 |

Ind. Individual Number

### Example 19

Light blue color Blue Springs and pale purple color flower Blue Springs were subjected to allogamous crossing, the seeds were seeded within the above-mentioned laboratory dish, the seedlings were raising and growing to obtain hybrid bicolor Blue Springs (bicolor light blue, B light blue) whose calyx was examined for anthocyanin. The results are shown in Table 13. Hybrid bicolor Blue Springs (bicolor light blue, B light blue) containing the novel anthocyanin (I) and also containing anthocyanin pigment (II) as a major pigment can be found to be obtained by the crossing. The anthocyanin pigment shown by (III) in Table 13 is a concentration of monodeacylcampanin. As intrinsic pigment in Table 13, 1 stands for bisdeacylplatyconin, 2 stands for tulipanin), 3 stands for violdelphin, and 4 stands for cyanodelphin.

TABLE 13

| | | CIELab | | | Intrinsic Pigment (nano mol/fresh weight g) | | | | | | |
|---|---|---|---|---|---|---|---|---|---|---|---|
| Blue Springs | Ind. | L* | C* | h | 1 | 2 | (III) | 3 | (I) | (II) | 4 |

Crossing Parent (Reciprocal Crossing)

| | | | | | | | | | | | |
|---|---|---|---|---|---|---|---|---|---|---|---|
| Light Blue | 1 | 62.3 | 32.6 | −62.2 | 0.0 | 0.0 | 28.4 | 71.8 | 10.6 | 31.4 | 399.5 |
| White | 1 | 72.1 | 17.5 | −52.7 | 0.0 | 2.2 | 13.4 | 680.3 | 22.7 | 6.1 | 0.0 |

Allogamous Crossing (Bicolor Blue)
Outside of Calyx

| | | | | | | | | | | | |
|---|---|---|---|---|---|---|---|---|---|---|---|
| Light Blue | 13 | 62.9 | 28.1 | −59.0 | 0.0 | 10.8 | 29.3 | 154.5 | 19.8 | 304.0 | 328.4 |

Inside of Calyx

| | | | | | | | | | | | |
|---|---|---|---|---|---|---|---|---|---|---|---|
| Pale Purple | 13 | 66.4 | 32.6 | −38.5 | 0.0 | 2.9 | 36.2 | 189.0 | 23.7 | 77.7 | 453.2 |

Ind. Individual Number

### Example 20

Light blue color Blue Springs and white color flower Blue Springs were subjected to allogamous crossing, the seeds were seeded within the above-mentioned laboratory dish, the seedlings were raising and growing to obtain hybrid bicolor Blue Springs (bicolor blue, B blue) whose calyx was examined for anthocyanin. The results are shown in Table 14. Hybrid bicolor Blue Springs (bicolor light blue, B light blue) containing the novel anthocyanin (I) and also containing anthocyanin pigment (II) as a major pigment can be found to be obtained by the crossing. The anthocyanin pigment shown by (III) in Table 14 is a concentration of monodeacylcampanin. As intrinsic pigment in Table 14, 1 stands for bisdeacylplatyconin, 2 stands for tulipanin), 3 stands for violdelphin, and 4 stands for cyanodelphin.

TABLE 14

| | | CIELab | | | Intrinsic Pigment (nano mol/fresh weight g) | | | | | | |
|---|---|---|---|---|---|---|---|---|---|---|---|
| Blue Springs | Ind. | L* | C* | h | 1 | 2 | (III) | 3 | (I) | (II) | 4 |

Crossing Parent (Reciprocal Crossing)

| | | | | | | | | | | | |
|---|---|---|---|---|---|---|---|---|---|---|---|
| Light Blue | 2 | 61.3 | 35.1 | −61.6 | 0.0 | 0.0 | 16.9 | 117.4 | 17.2 | 22.8 | 380.4 |
| White | 1 | 81.8 | 3.3 | −30.2 | 776.4 | 0.0 | 0.0 | 0.0 | 0.0 | 0.0 | 0.0 |

Allogamous Crossing (Bicolor Blue)

| | | | | | | | | | | | |
|---|---|---|---|---|---|---|---|---|---|---|---|
| Outside of Calyx | | | | | | | | | | | |
| Blue | 2 | 46.2 | 70.0 | −49.4 | 0.0 | 8.1 | 37.9 | 124.6 | 23.9 | 120.1 | 390.2 |
| Inside of Calyx | | | | | | | | | | | |
| Purple | 2 | 53.7 | 53.5 | −33.8 | 6.0 | 4.7 | 49.8 | 179.8 | 29.9 | 234.7 | 305.8 |

Ind. Individual Number

### Example 21

Light blue color Pacific Giant and red pink color flower Blue Springs were subjected to allogamous crossing, the seeds were seeded within the above-mentioned laboratory dish, the seedlings were raising and growing to obtain purple flower color delphinium hybrid variety whose calyx was examined for anthocyanin. The results are shown in Table 15. The purple flower color delphinium hybrid variety containing the novel anthocyanin (I) and also containing anthocyanin pigment (II) as a major pigment can be found to be obtained by the crossing. The anthocyanin pigment shown by (III) in Table 15 is a concentration of monodeacylcampanin. As intrinsic pigment in Table 14, 1 stands for bisdeacylplatyconin, 2 stands for tulipanin), 3 stands for violdelphin, and 4 stands for cyanodelphin.

TABLE 15

| | | CIELab | | | Intrinsic Pigment (nano mol/fresh weight g) | | | | | | |
|---|---|---|---|---|---|---|---|---|---|---|---|
| Blue Springs | Ind. | L* | C* | h | 1 | 2 | (III) | 3 | (I) | (II) | 4 |

Crossing Parent (Reciprocal Crossing)

| | | | | | | | | | | | |
|---|---|---|---|---|---|---|---|---|---|---|---|
| Pacific Giant | | | | | | | | | | | |
| Pale Purple | 1 | 58.7 | 31.1 | −46.2 | 0.0 | 0.0 | 47.5 | 340.5 | 45.6 | 463.6 | 0.0 |
| Blue Springs | | | | | | | | | | | |
| Red Pink | 1 | 55.8 | 35.7 | −22.0 | 1198 | 2434 | 0.0 | 0.0 | 0.0 | 0.0 | 0.0 |

Allogamous Crossing

| | | | | | | | | | | | |
|---|---|---|---|---|---|---|---|---|---|---|---|
| Purple | 6 | 29.2 | 78.9 | −36.2 | 28.8 | 38.3 | 425.6 | 1122 | 139.6 | 1749 | 0.4 |

Ind. Individual Number

### Example 22

Light blue color Pacific Giant and blue color flower Blue Springs were subjected to allogamous crossing, to obtain pale purple flower color delphinium hybrid variety containing the novel anthocyanin (I) and also containing anthocyanin pigment (II) as a major pigment can be found to be obtained by the crossing. The anthocyanin pigment shown by (III) in Table 16 is a concentration of monodeacylcampanin. As intrinsic pigment in Table 16, 1 stands for bisdeacylplatyconin, 2 stands for tulipanin), 3 stands for violdelphin, and 4 stands for cyanodelphin.

TABLE 16

| | | CIELab | | | Intrinsic Pigment (nano mol/fresh weight g) | | | | | | |
|---|---|---|---|---|---|---|---|---|---|---|---|
| Blue Springs | Ind. | L* | C* | h | 1 | 2 | (III) | 3 | (I) | (II) | 4 |

Crossing Parent (Reciprocal Crossing)

| | | | | | | | | | | | |
|---|---|---|---|---|---|---|---|---|---|---|---|
| Pacific Giant | | | | | | | | | | | |
| White | 1 | 82.1 | 3.5 | −36.1 | 628.7 | 0.0 | 0.0 | 0.0 | 0.0 | 0.0 | 0.0 |
| Blue Springs | | | | | | | | | | | |
| Blue | 1 | 42.2 | 74.0 | −49.5 | 11.6 | 7.0 | 50.3 | 420.7 | 65.1 | 33.7 | 822.1 |

Allogamous Crossing

| | | | | | | | | | | | |
|---|---|---|---|---|---|---|---|---|---|---|---|
| Purple | 1 | 65.0 | 30.2 | −46.5 | 0.0 | 14.0 | 9.3 | 152.8 | 28.9 | 179.3 | 0.0 |

Ind. Individual Number

From these Examples, the method for crossing delphinium based on flower color according to the present invention is proven to be an excellent method for producing delphinium allowing a specific flower color to be inherited to a progeny, bicolor delphinium, and delphinium having flower color realized by novel anthocyanin.

### INDUSTRIAL APPLICABILITY

Delphinium is an allogamous plant and thus, causes self-propagation weakness when repeating self-propagation. Consequently, it is difficult to propagate seeds having a specific flower color, seeds having specific flower color cannot be maintained. The present invention allows delphinium for allogamous crossing to inherit a specific flower color to a progeny.

In conventional it is difficult to allow delphinium for effectively growing at a warm place, but the present invention makes it possible to effectively growing at a warm place by germinating such conditions when delphinium undergoes allogamous crossing to thereby allow a specific flower color to be inherited to a progeny.

Conventionally, Bicolor delphinium can only be propagated by mericlone seedlings, and it is not possible to make a seed propagation. By the allogamous crossing of the present invention, Conventionally, the delphinium is well growing at a cool or cold place or a high and cold place, and is not well growing at a warm place unless it is forcedly cultivated. When raising seedling under the conditions that delphinium which has undergone allogamous crossing is germinated within a laboratory dish at a temperature of approximately 15° C., delphinium can be effectively growing (at a season), and particularly at a warm place.

Conventionally, the relation between flower color of calyx of delphinium and intrinsic pigment within the calyx. According to the present invention, the flower color of delphinium can be determined from the ratio of major intrinsic pigment within the calyx.

According to the present invention, a method is provide, which undergoes allogamous crossing of delphinium having whole color type flower color to thereby obtain seeds having a specific flower color. At the same time, a method is provided, which can obtain seeds of delphinium having bicolor flower color by allogamous crossing. Also, the present invention provide a method for effectively cultivating delphinium at a season at a warm place.

According to the present invention, delphinium having bicolor flower color from seed propagation.

According to the present invention, a novel anthocyanin pigment can be provided from delphinium.

1. A method for crossing delphinium based on flower color comprising
utilizing delphinium having whole color type flower color as a pollen parent or a seed parent to allogamous crossing to thereby allow a specific flower color to be inherited to a progeny.

2. The method for crossing delphinium based on flower color according to claim 1, wherein said specific flower color is bicolor flower color.

3. A method for cultivating delphinium comprising raising seedling under the conditions that delphinium is germinated within a laboratory dish at a temperature of 15° C.±1° C.

4. A method for determining flower color of delphinium which comprising applying an equation 1:
$$H = {H}_{max} \frac{[ CD / VD ]}{[ CD / VD ] + {K}_{H}} = {tang}^{- 1} ( {b}^{*} / {a}^{*} ) \qquad ( 1 )$$
where ratio of content of intrinsic pigment within the calyx [CD/VD] is taken as a horizontal axis and hue angle exhibiting flower color
wherein CD/VD exhibits ratio of major intrinsic pigment within the calyx of delphinium; CD is Cyanodelphin, VD is of Violdelphin, Hmax is the maximum hue angle showing flower color, and K_{H} is a constant of ratio of intrinsic pigment in the case of half of the maximum, hue angle.

5. The method for determining flower color of delphinium according to claim 4, which applies an equation [VD/TP] to [CD/VD]
wherein [VD/TP] is a concentration ratio of major intrinsic pigment, and calculated by dividing Violdelphin concentration VD by tulipanin concentration TP.

6. A method for crossing delphinium based on flower color according to claim 1 or 2, which determines the combination of flower color crossing for creating flower color of delphinium, which assumes flower color utilizing a simplified chart taking gamete of pollen parent or seed parent as a line or column.

7. A method for crossing delphinium according to claim 1 or 2, which determines flower color of delphinium based on predetermined anthocyanin pigment.

8. The method for crossing delphinium according to claim 7, wherein the predetermined anthocyanin pigment is a novel anthocyanin pigment, 3-O-(6-O-(α-L-rhamnosyl)-β-D-glucopyranosyl)-7-O-(3-O-β-D-glucopyranosyl-6-O-(4-O-(6-O-p-hydroxybenzoyl-β-D-glucopyranosyl)-p-hydroxybenzoyl)-β-D-glucopyranosyl)-delphinidin represented by the following formula (I):

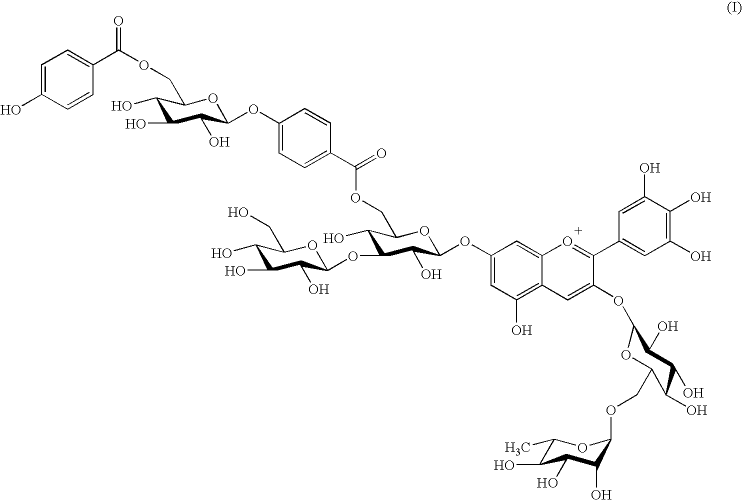

9. The method for crossing delphinium according to claim 7, wherein the predetermined anthocyanin pigment is an anthocyanin pigment, 3-O-(6-O-(α-L-rhamnosyl)-β-D-glucopyranosyl)-7-O-(3-O-(3-O-(β-D-glucopyranosyl)-β-D-glucopyranosyl)-6-O-(4-O-(6-O-p-hydroxybenzoyl-β-D-glucopyranosyl)-p-hydroxybenzoyl)-β-D-glucopyranosyl)-delphinidin(II) represented by the following formula (II):

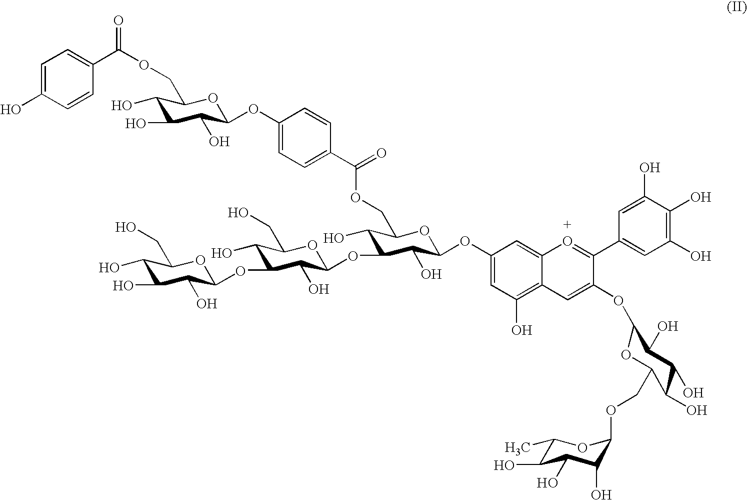

10. A method for extracting an anthocyanin pigment which comprises isolating anthocyanin pigment (I), anthocyanin pigment (II), or both anthocyanin pigments them from the calyx of delphinium obtained by the method The method for crossing delphinium according to claim 8 or 9, followed by purifying.

11. A novel compound, 3-O-(6-O-(α-L-rhamnosyl)-β-D-glucopyranosyl)-7-O-(3-O-β-D-glucopyranosyl-6-O-(4-O-(6-O-p-hydroxybenzoyl-β-D-glucopyranosyl)-p-hydroxybenzoyl)-β-D-glucopyranosyl)-delphinidin, represented by the following formula (I): [chemical structure]
